(19) United States
(12) Patent Application Publication
(10) Pub. No.: US 2024/0248008 A1
(43) Pub. Date: Jul. 25, 2024

(54) METHODS, DEVICES, AND SYSTEMS FOR AEROSOL DETECTION

(71) Applicant: N5 SENSORS, INC., Rockville, MD (US)

(72) Inventors: Abhishek MOTAYED, Bethesda, MD (US); Brian THOMSON, Washington, DC (US); Rudy VILLEGAS, Rockville, MD (US); Dimitri ALEXSON, Fairfax, VA (US)

(21) Appl. No.: 18/421,900

(22) Filed: Jan. 24, 2024

Related U.S. Application Data
(60) Provisional application No. 63/481,362, filed on Jan. 24, 2023.

(51) Int. Cl. G01N 1/22; G01N 1/02; G01N 1/24

(52) U.S. Cl. CPC G01N 1/2202; G01N 1/2273; G01N 2001/022; G01N 2001/2223; G01N 2001/2276; G01N 1/24

(57) ABSTRACT

Methods, devices, and systems for aerosol detection, such as NTAs and PBAs are disclosed. The device can be worn, handheld, or mounted to an apparatus.

### RELATED APPLICATION

This application is related to, and claims priority from U.S. Provisional Patent Application No. 63/481,362, filed on Jan. 24, 2023, entitled “ChemBadgER: A Person Worn Chemical Aerosol Detector,” the entire disclosure of which is incorporated here by reference.

### BACKGROUND

The nature and number of chemical hazards have grown significantly over the last 20 years. The concept of “weapons of mass destruction” (WMD) has been supplanted to some degree by the concept of weapons of mass disruption and casualty. Common chemicals such as acids, bases, accelerants, and a host of pharmaceuticals are of growing concern as they can be procured by non-State actors and readily deployed by unskilled individuals. The nature of the threat posed by chemical warfare agents is similarly evolving with the development of dusty and binary agents, and of next-generation, lower volatility materials referred to as Non-Traditional Agents (NTAs). The chemical threat scenario has similarly expanded to include the ubiquitous opioids and in particular illegally procured or manufactured opioids. Collectively, these threats are known as pharmaceutical-based agents (PBAs), with the fentanyl family of compounds being the most available, widespread, and lethal. These PBAs are prevalent and available on the streets and pose a threat from simple inhalation or contact with a person. For example, fentanyl (parent compound) is a synthetic opioid that is 50 to 100 times more potent than heroin and morphine, respectively. A related compound, carfentanil, is about 100 times more potent than fentanyl. Illicitly manufactured fentanyl (IMF) is available on the drug market in different forms, including liquid and powder. This evolving threat scenario is complicated since these materials (e.g., next-generation chemical threats, PBAs) are low volatility chemicals that can be easily disseminated as a liquid and dry aerosol.

Examples of chemical vapor detectors are shown in Table 1. Vapors are Toxic Industrial Chemicals (TICs) and Chemical Warfare Agents (CWAs). In the table below, LEL is the lower explosive limit.

TABLE 1
Examples of chemical vapor detectors

| Device | Threats | Technology | Sampling | Mission Use | Challenges |
|---|---|---|---|---|---|
| JCAD/LCD3 | CWAs | IMS | Vapors, | Portable, Real- | TIC Library, |
| [symbol] | PBAs, | | Solid/ | time detection/ID | False alarms, |
| | NTAs, | | Liquid | for CWAs and | consumables |
| | Some | | Samples | some TICs. | |
| | TICS | | with | Solid/liquid | |
| | | | Adaptor | Adapter | |
| | | | | attachment is | |
| | | | | new add on. | |
| MX908 | CWAs, | HPMS | Vapor | Portable, Real- | Large, |
| [symbol] | PBAs, | — | — | time detection/ID | expensive |
| | NTAs | HPMS | Aerosol | of Solid, Liquid | |
| | | Aero | | Aerosols, and | |
| | | | | Vapor | |
| Hapsite | CWAs, | GC/MS | Vapors | Portable, Site | Large (40 lbs.), |
| [symbol] | PBAs, | | and | Survey, | expensive, |
| | NTAs, | | Solid | remediation, | requires |
| | TICS | | phase | confirmatory | consumables |
| | | | Micro- | analysis | and frequent |
| | | | extraction | | maintenance |
| MultiRAE | LEL, | PID, EC | Vapors | Hand-held, | Limited amount |
| [symbol] | O₂, up | | | Real-time | of sensors (small |
| | to 5 | | | detection/ID | library) for a |
| | TICS | | | device for TIC, | particular |
| | | | | LEL, and O₂ | configuration, |
| | | | | vapor threats | selectivity, sensor |
| | | | | | lifetime. Vapor |
| | | | | | threats only |
| N5 | TICS, | Semi- | Vapors | Wearable | Vapor threats |
| ChemBadge | CWAs, | conductor | | Real-time | only |
| [symbol] | O₂, | Photo | | detection/ID | |
| | LEL | catalytic | | device for TIC, | |
| | | Hybrid | | CWA, LEL, and | |
| | | (SPH) | | O₂ vapor threats | |

Most of the devices listed in Table 1 detect only chemical vapors. There has been research and development work done over the last 10 years using novel materials and structures such as metal-organic frameworks (MOFs), 2-dimensional and 3-dimensional graphene, graphene tubules (carbon nanotubes), and nanofibers as sensing substrates. Graphene-based sensors are prone to environmental instability related to oxidative reactions. Polymer-based coatings or nanofibers can similarly exhibit changes due to humidity and temperature. Some companies are developing chemical aerosol detection capabilities, but these systems are projected to be greater than 10 pounds in weight, and with a size not compatible with a person-worn device.

The chemical detectors currently in use by the Joint Services, related Government agencies, and civilian emergency responders are generally designed to detect chemical hazards, which present as vapors or gases. Thus, current devices are not equipped to detect lower-volatility agents and chemicals disseminated in aerosol form. There are developmental efforts, such as under the U.S. Department of Defense's Next Generation Chemical Detector (NGCD) and Aerosol Vapor Chemical Agent Detector (AVCAD), currently underway. Yet, these efforts are anticipated to result in instrumentation that is 10 pounds or greater in weight. While AVCAD development efforts may result in a very capable new chemical detector, these technologies are not compatible with a person-worn chemical threat detector configuration. Optical spectroscopy, including infrared (IR), Raman, fluorescence, and a multitude of variants based on these (e.g., Surface Enhanced Raman, Mid/Near-IR, Laser Induced Fluorescence) are often employed to detect a wide range of chemical threats both in the laboratory and as hand-held field instruments. These instruments are well-suited to first responder situations and “white powder” incidents with liquid or unknown solids, typically present in bulk form. Two primary drawbacks are their inability to specifically detect aerosolized chemical threats and the challenges of implementation in a truly person-worn configuration. Compounding the challenge is the need to detect these chemical threats in a complex ambient background, including the urban and maritime landscapes. This requires innovation not only on the sensor and detection front, but also on the data processing methods. Particularly vulnerable in this new threat scenario are military and civilian emergency responders and law enforcement personnel.

Thus, to solve the current issues above, embodiments disclosed herein provide methods, devices, and systems which enable a user (e.g., emergency responders) to wear a small-size sensing device (detector) to sense aerosols, such as liquid and solids, which are converted to a gas phase by the sensing device. The sensing device that can be worn by a user is no larger than the size of an average cell phone or bodycam. In an embodiment, the sensing device is 8 cm×5 cm×1 cm, with a weight of less than 100 grams. The aerosols can be chemical or biological. The device can detect minute amounts of aerosol, and the sensing device acts in real-time to give a user immediate information and alerts about any possible threats. The sensing device could be handheld or mounted to an apparatus, such as a building, a pole, a drone, etc. When mounted or attached to an apparatus, emergency responders, for example, are not harmed by hazardous substances detected by the sensing device.

Unlike some of technologies listed in Table 1, the embodiments herein offer several advantages as the detector already meets several of the requirements for a person-worn device. For example, the detector is augmented with advanced data processing, there is no secondary receptor technology required, it is easy to add new explosive chemicals/precursors to the sensor array, it includes high-density arrays in a micro-footprint, and the semiconductor fabrication is low-cost, with a rapid scale-up. Examples of secondary receptor technology include biological assays, where biological threats are captured and then transferred to receptor sites. In this case, the collection, receptor and detection technology are all integrated.

### SUMMARY

According to an embodiment, a method for detecting an aerosol is disclosed, wherein the method comprises: collecting aerosol particles; heating the aerosol particles; converting the aerosol particles to a vapor; sending the vapor to at least one sensor; analyzing the vapor; and determining an aerosol type.

According to an embodiment, a device for detecting an aerosol is disclosed, wherein the device comprises: an aerosol collector to collect aerosol particles; a heater to heat the aerosol particles; and a computer processor configured to: convert the aerosol particles to a vapor; send the vapor to at least one sensor; analyze the vapor; and determine an aerosol type.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1A illustrates a microfabricated sensor die;

FIG. 1B illustrates a packaged sensor device;

FIG. 1C illustrates a sensor module with the embedded sensor device;

FIG. 1D illustrates the fully integrated detector system;

FIG. 2 illustrates the different techniques used for control of the SPH sensor functional material properties;

FIG. 3 illustrates SPH gas sensor technology;

FIG. 4 illustrates the basic components of the SPH array technology;

FIG. 5 illustrates a sensor array architecture;

FIG. 6 illustrates simulants according to an embodiment;

FIG. 7 illustrates a sensing mechanism according to an embodiment;

FIG. 8 illustrates a detection according to a sensor array;

FIG. 9 illustrates a detection according to a sensor array;

FIG. 10 illustrates further potential simulants;

FIG. 11 illustrates a microchannel collector;

FIGS. 12A and 12B illustrate a microwell collector;

FIG. 13 illustrates an aerosol detection device according to an embodiment;

FIG. 14 illustrates an aerosol detection device according to an embodiment;

FIG. 15 illustrates a microheater device used in an embodiment;

FIG. 16A illustrates the SPH sensor die and microheater device arranged onto the surface of a Peltier Element according to an embodiment;

FIGS. 16B and 16C illustrate the aerosol sensing configuration according to an embodiment;

FIG. 17 illustrates a microheater/SPH sensor die component according to an embodiment;

FIG. 18 illustrates a method of the breadboard testing using the microwell aerosol collector according to an embodiment;

FIG. 19 illustrates the detection of malathion according to an embodiment;

FIG. 20 illustrates tight control of particle size and layer thickness;

FIG. 21 illustrates an electric field microscopy (AFM) for dispersion estimation and film thickness;

FIG. 22 illustrates a block diagram according to an embodiment;

FIG. 23 illustrates a device using the microwell collector according to an embodiment;

FIG. 24 illustrates a device using the microchannel collector according to an embodiment;

FIG. 25 illustrates circuitry for a device;

FIG. 26 illustrates a sensing device according to an embodiment;

FIG. 27 illustrates a method according to an embodiment;

FIG. 28 illustrates a method according to an embodiment;

FIG. 29 illustrates a sensing device according to an embodiment;

FIG. 30 illustrates an algorithm according to an embodiment;

FIG. 31 illustrates a confusion matrix for gas identification according to an embodiment;

FIG. 32 illustrates a plot of the aerosol concentration vs time in the dilution chamber and the exposure chamber;

FIG. 33 illustrates a method according to an embodiment;

FIG. 34 illustrates a device according to an embodiment; and

FIG. 35 illustrates a device according to an embodiment.

### DETAILED DESCRIPTION

The embodiments are more fully described herein with reference to the accompanying drawings, in which embodiments of the inventive concept are shown. In the drawings, the size and relative sizes of layers and regions may be exaggerated for clarity. Like numbers refer to like elements throughout. The embodiments may, however, be embodied in many different forms and should not be construed as limited to the embodiments set forth herein. Rather, these embodiments are provided so that this disclosure will be thorough and complete, and will fully convey the scope of the inventive concept to those skilled in the art. The scope of the embodiments is therefore defined by the appended claims.

Reference throughout the specification to “one embodiment” or “an embodiment” means that a particular feature, structure or characteristic described in connection with an embodiment is included in at least one embodiment of the present invention. Thus, the appearance of the phrases “in one embodiment” or “in an embodiment” in various places throughout the specification is not necessarily all referring to the same embodiment. Further, the particular features, structures or characteristics may be combined in any suitable manner in one or more embodiments.

As used herein, the singular forms “a”, “an”, and “the” are intended to include the plural forms as well, unless the context indicates otherwise. In the specification and the claims, the term “and/or” is intended to include any combination of the terms “and” and “or” for the purpose of its meaning and interpretation.

Some embodiments herein relate to N5's ChemBadge—Emergency Response (ChemBadgER), which is a person-worn chemical aerosol detector capable of real-time detection of multiple classes of chemical threats. The ChemBadgER is based on proprietary Semiconductor Photocatalytic Hybrid (SPH) sensing technology that enables continuous monitoring and semi-quantification of environmental and hazardous gases. An SPH sensor array is discussed in N5's International Patent Publication No. WO 2023/043428. An integrated micro-scale aerosol collector (IMAC) can interface with the SPH sensor array. The ChemBadgER expands this capability to detect both chemical threats in the gas phase, and also those presented as liquid or solid aerosols. An aerosol is a suspension of fine solid particles or liquid droplets in air or another gas. Aerosols can be natural or anthropogenic. The term aerosol commonly refers to the particulate/air mixture, as opposed to the particulate matter alone. Examples of natural aerosols are fog or mist, dust, forest exudates, and geyser steam. Examples of anthropogenic aerosols include particulate air pollutants, mist from the discharge at hydroelectric dams, irrigation mist, perfume from atomizers, smoke, dust, steam from a kettle, sprayed pesticides, and medical treatments for respiratory illnesses. Systems that can detect gas vapors works with individual molecules of gas reaction. In comparison, aerosol detection systems need to capture the aerosol particle (which could be solid or liquid) and then analyze it. Many of the new and evolving chemical threats are low volatility chemicals most amenable to dissemination as an aerosol. An additional benefit of the N5's sensor technology is that the SPH sensor technology benefits from a design “toolkit” enabling rapid development of new sensors and “fit-for-purpose” capability. Thus novel, emerging chemical threats can be responded to quickly through additions to the detector library. In addition, sensing devices can be customized for desired uses.

A semiconductor photocatalytic hybrid (SPH) vapor sensor array technology can be used to detect new chemical threats beyond the proven detection levels for toxic industrial chemicals (TICs), chemical warfare Agents (CWAs), and environmental gases (e.g., NOx, H2S, NH3, SO2, O3). The NTAs and PBAs represent classes of emerging chemical threats that are much lower in volatility than some of the TIC or CWA threats, and which would be disseminated in liquid or solid aerosol form. Embodiments herein discuss sensor technology, which is able to (1) detect aerosolized chemicals and (2) detect the new classes, such as NTAs and PBAs, which are a threat. In addition, sensor technology herein uses small, high-efficiency aerosol sampling technology including: inlets, collectors, desorbers, transfer designs, and methods. In some embodiments, an aerosol sampling device can detect particles in the respirable 0.5-10 micron range. Some embodiments include: detection of chemical aerosols, which may be low-volatility, such as NTA and PBA simulants with the SPH sensor array; demonstration of aerosol capture of liquid and solid aerosols; and desorption of a target chemical simulant and detection with the SPH array. In some embodiments, the sensing device includes an efficient aerosol inlet and sampler, which is designed to be a small size, low weight, but with enough power, so that it is suitable to be worn by a person or used as a handheld device.

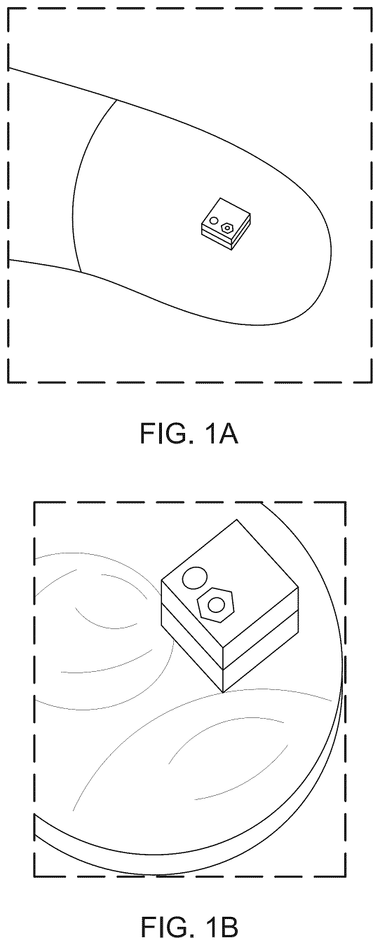

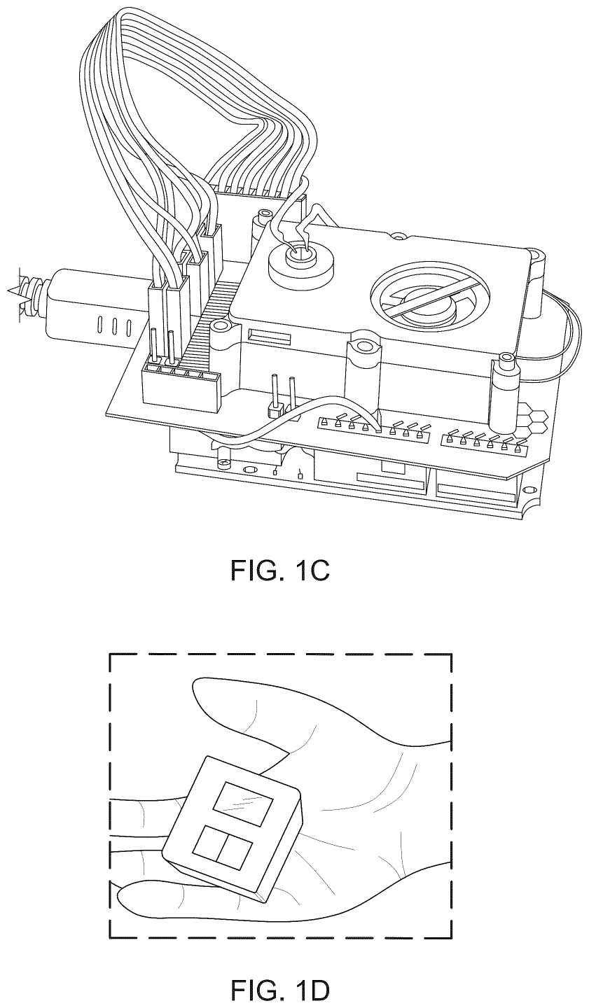

N5 has developed patented microscale chemical sensor technology, which allows for real-time detection of chemical vapors including toxic industrial chemicals (TICs), Chemical Warfare Agents (CWAs), and other environmental gases. The sensors and products containing the sensors can be configured as small handheld instruments or person-worn badges. For example, FIG. 1A illustrates a microfabricated sensor die, to show the size with respect to a human finger. The size of the sensor die is very small. One sensor die is about 1×1 square mm currently. An array of six sensor dies is approximately 1×1 inch. FIG. 1B illustrates a packaged sensor device with respect to a U.S. penny to indicate the size of the packaged sensor device. FIG. 1C illustrates a sensor module with the embedded sensor device. The sensor module has dimensions of about 8 cm×6 cm×1 cm, a weight of 0.4 pounds, and power consumption requirements for continuous operation of about 0.2 watts, with multiple active sensor elements or channels for simultaneous measurement of gases. FIG. 1D illustrates the fully integrated detector system, which can be handheld, worn by a user, or mounted to an apparatus. The fully integrated detector system provides a display screen so that a user can easily know if certain levels are detected.

The patented Semiconductor Photocatalytic Hybrid (SPH) platform combines high-performance photoconductors with metal-oxide photocatalytic nanoclusters, resulting in selective microsensors on a chip. The architecture represents a paradigm shift in low-power, compact chemical sensors, and addresses the capability gap that exists for small size, weight, and power (SWaP) detectors and current mature sensor technologies that are used in handheld systems. Fielded systems and some developmental sensor technologies suffer from one or more of the following: poor molecular selectivity and false positive or negative responses; fair sensitivity; environmental instability; toxicity of materials; and high cost of materials or fabrication. Equally important, some currently fielded chemical sensors cannot be rapidly upgraded to add new and emerging threats including materials such as NTAs and PBAs. Thus, embodiments are specifically designed to address these current limitations.

### SPH Sensor Array Fabrication and Quality Control

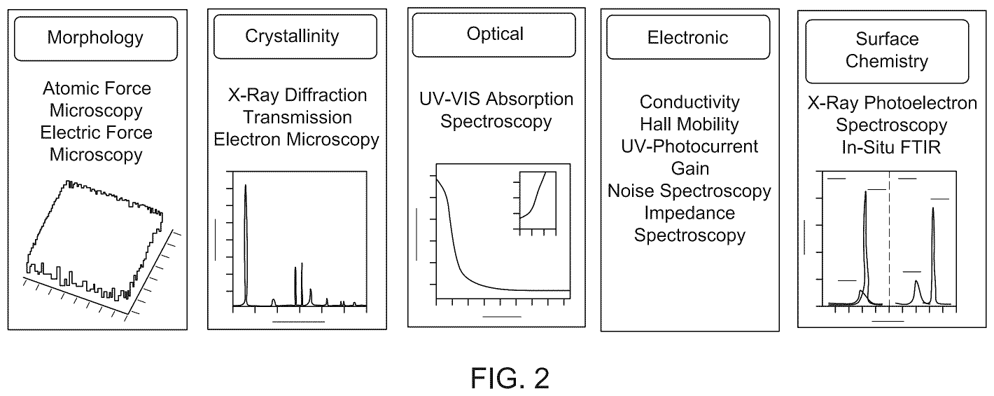

FIG. 2 shows the different techniques used for control of the SPH sensor functional material properties. Over the years, N5 has developed expertise in controlling the functional material with wafer-scale reproducibility and die level tracking. N5's commercial products utilize such process control measurements to ensure wafer-to-wafer control.

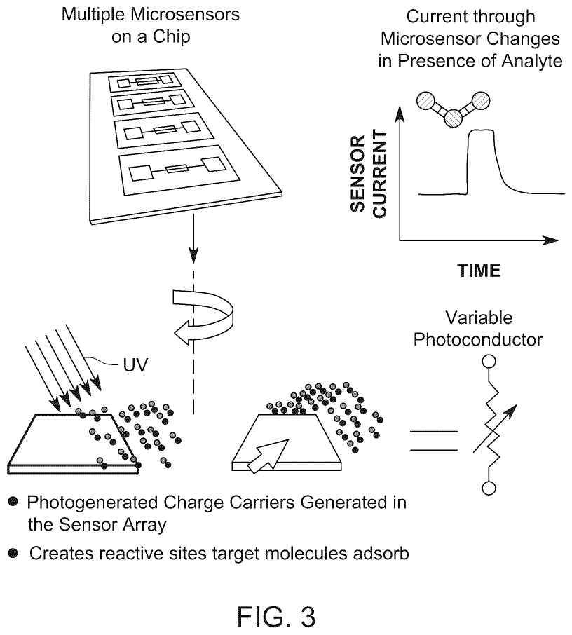

As shown in FIG. 3, N5's SPH gas sensor technology employs metal oxides as the receptor, which provides the reactive sites for target molecule adsorption when excited with UV light. The challenges associated with conventional metal oxide (MOx) sensors are well-known, including the requirement for high-temperature heating, and sensitivity and selectivity limitations. N5's SPH sensor technology does not require elevated temperatures for operation, but rather relies on “photoexcitation” using a small, on-chip light emitting diode (LED).

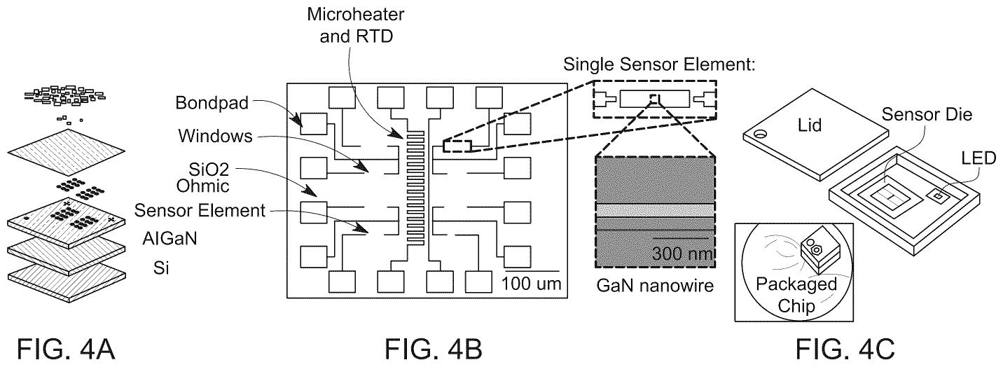

The basic components of the N5's SPH array technology are shown in FIG. 4. FIG. 4 illustrates: (a) the sensor's construction; (b) the sensor substrate; and (c) the integrated sensor. The sensor package consists of a gallium nitride, GaN-on-Si sensor die along with a UV LED die in a single package. N5 has developed the sensor packaging to integrate all of the sub-components of the sensor technology inside a small form factor surface-mountable package. The N5 chemical sensors are also unique in that metal co-catalysts have been developed and tested to impart specific properties in terms of selectivity, sensitivity, dynamic range, and response time. This design toolkit enables not only fit-for-purpose detection solutions, but also a rapid and agile response to the detection of new, evolving threat agents.

N5's SPH sensor array technology combines a large number of parallel SPH sensing elements with unique material properties. The SPH sensor arrays can be configured as low, medium, or higher density arrays (i.e., the number of individual sensor elements in the array is scalable) to meet specific detection needs. These sensor arrays may be used for the detection and identification of TICs and CWAs. The arrays provide rich data sets for identifying diverse molecules in complex environments. N5 has developed advanced pattern recognition algorithms which can analyze the multichannel data for real-time warning and reporting. New threats can be rapidly added to detection libraries using materials optimization and flexible algorithms.

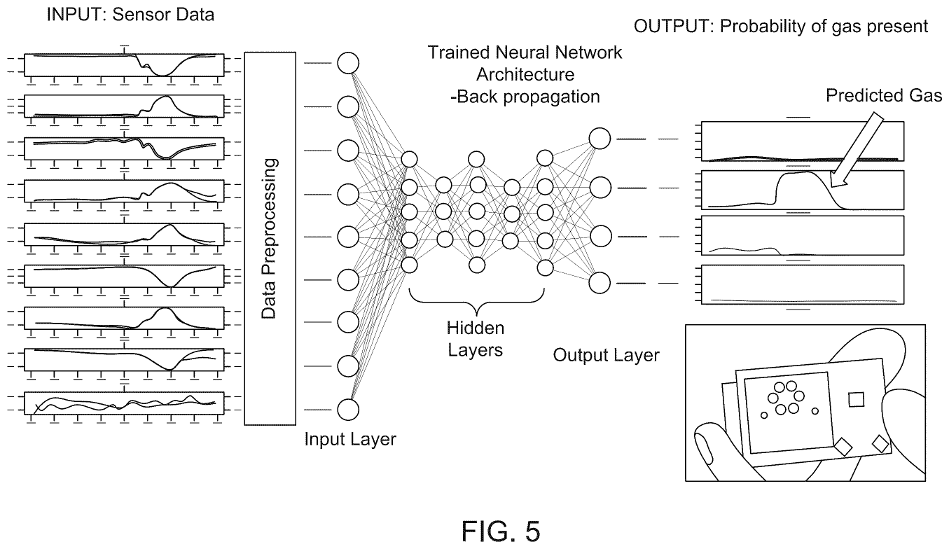

The sensor array architecture enables the application of advanced algorithms and machine learning principles for vapor identification, as shown in FIG. 5, for example. A sensor can communicate with an edge cloud or another computing device. In addition, each sensor can communicate with other sensors in other locations, where the data can be accumulated, stored, and communicated in real-time to others. The SPH sensors are configured in an array, where each sensor is designed with different materials to impart unique sensing performance. The array data is analyzed as an ensemble to produce the detection output. This approach improves selectivity and is a discriminator for the development of a multi-threat chemical agent detector. As an example, N5 developed an array of toxic gas sensors for detecting analytes (e.g., NH3, H2S, CI2, O2) integrated into a wearable gas detector system. A neural network was trained on the input data to identify gas threats based on unique response characteristics. The algorithm was implemented on the device and was validated against a blind dataset to demonstrate the correct prediction of target analytes.

### Detection of Chemical Aerosols

The SPH sensor arrays have been developed by N5 for the detection of chemical vapors. However, embodiments herein disclose the capabilities to expand the SPH sensor array for the detection of the lower volatility CWAs, as well as aerosols, such as NTAs and PBAs. In particular, the threat scenario being addressed is the dissemination of these agents as either liquid or dry aerosols. Detection of aerosolized chemical threats remains a capability gap for the Department of Homeland Security (DHS), its related organizations, and the Department of Defense (DOD). Some new technology development efforts funded by DHS have focused on the sampling of particulates off surfaces for use in conjunction with Explosive Trace Detection (ETD) systems. The sampling of aerosolized biological agents (e.g., bacteria, spores, and viruses) has been based on cyclonic and inertial impaction technologies. Current global health concerns have highlighted the need for improved efficiency in the sampling and collection of virus particles. Chemical aerosol sampling and detection to date have typically been based on the deposition of the aerosol onto a substrate (polymer resins, metals/metal meshes, wire filaments) and then typically heating those elements to release the chemical material as a vapor which then is sent to a detector. Other approaches are based on imparting an electric charge (e.g., via a corona discharge) to the aerosols and then capturing them on a collection plate. However, the detection of low-volatility CWAs, NTAs, and PBAs with a small, wearable device, remains an unmet need.

An aspect of the development of a person-worn detector for aerosolized chemical threats includes researching and evaluating approaches to the sampling, capture, and detection of chemical threats when disseminated in both liquid and dry aerosol form. The aerosol sampler should be compatible in size, weight and power with the sensor array to realize this goal. The ChemBadgER consists of a micro-aerosol sampler, integrated with the SPH sensor array in a small wearable device. ChemBadgER enables the detection of multiple classes of threat including TICs, CWAs, NTAs, and PBAs providing a unique capability that does not currently exist. In addition, the fundamental nature of the SPH sensors and supporting algorithms make it relatively easy and fast to add new, emerging threats to the detection library.

### Semiconductor Photocatalytic Hybrid (SPH) Sensor Design

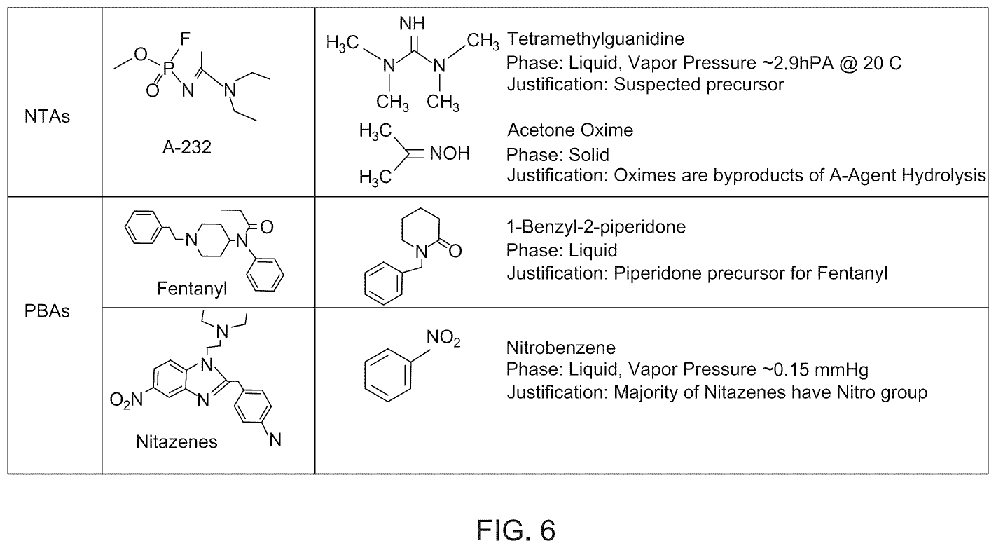

N5 compiled a list of NTA and PBA agents, chemical structures, and chemical properties (where available). At least three target and simulant chemicals were identified and tested: NTA (A-232), PBA (Fentanyl), and, PBA (Nitazene). FIG. 6 illustrates the simulants for the NTAs and PBAs that were used for laboratory demonstration, testing, and evaluation.

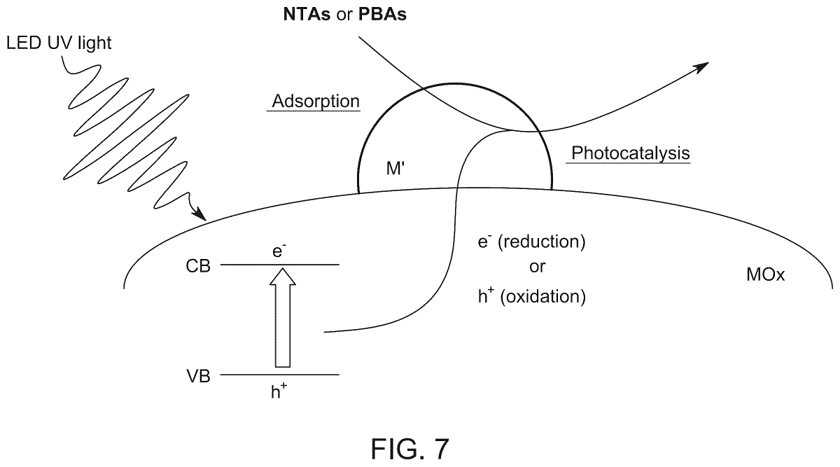

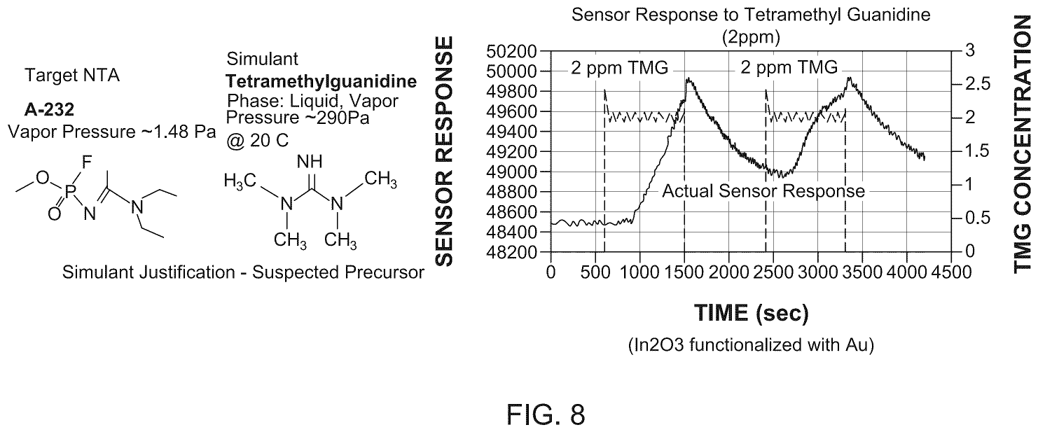

Plausible reaction pathways for the interaction of the SPH sensors with A-232 and Fentanyl were developed. As shown in FIG. 7, the sensing mechanism of NTAs or PBAs on the surface of the SPH sensor is illustrated. FIG. 7 depicts the SPH sensing mechanism in the presence of targeted chemicals. The photocatalysis-assisted sensing involves two primary surface reactions: 1) adsorption of target analytes, and 2) photocatalytic conversion of adsorbed chemicals. The surface of N5's semiconductor photocatalytic hybrid (SPH) sensors consists of two functional layers: a semiconductor metal oxide (MOx) photocatalyst (e.g., TiO₂, ZnO, SnO₂, etc.) and metal (M′) cocatalysts (e.g., Pt, Au, Pd, etc.). The targeted chemicals are typically adsorbed at the interfacial region between MOx and M′, and subsequently, the adsorbed chemicals are photo-catalytically converted to oxidants (sensor accepting electrons) or reductant (sensor providing electrons). The significance is that interfacial carrier transfer between chemicals and devices happens only when the targeted chemical is adsorbed on the sensing layer. Therefore, selecting combinations of MOx and M′ is most critical to develop high-performance SPH sensors in terms of the adsorption of desired analytes and subsequent carrier transfer between the sensing layer and analytes.

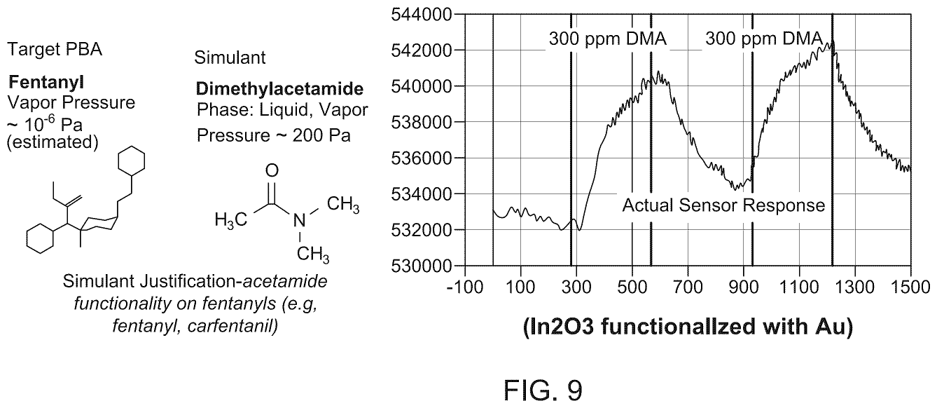

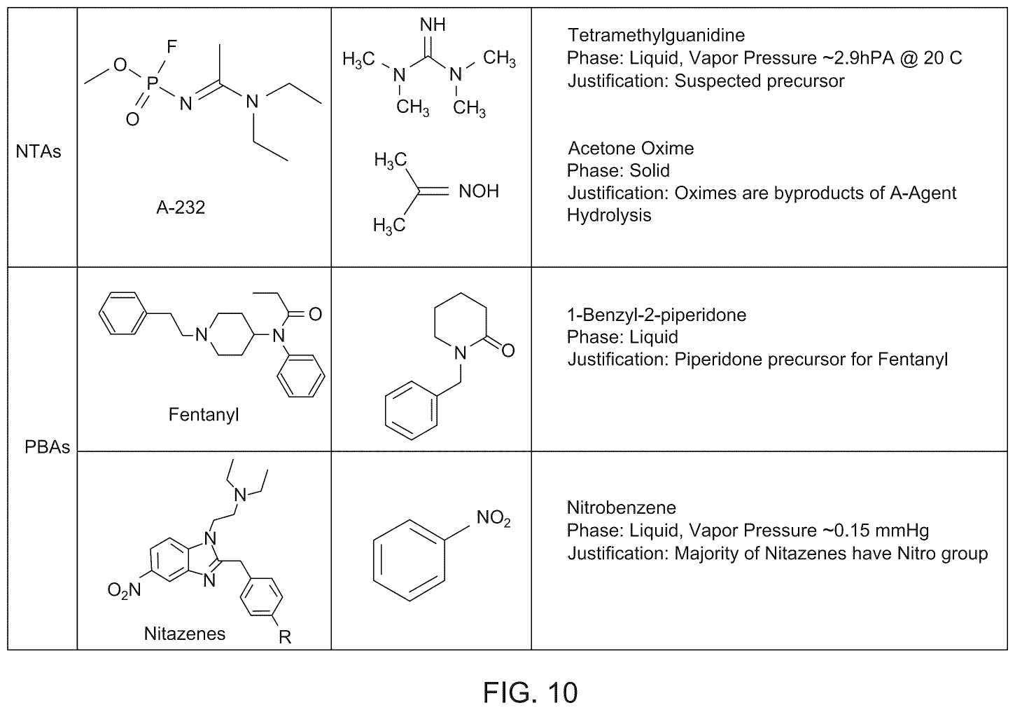

Tested simulants for the NTAs and PBAs with an existing SPH sensor array are shown in FIGS. 8 and 9. As shown in FIGS. 8 and 9, two simulants are detected, Tetramethylguanidine (NTA simulant) and Dimethylacetamide (PBA simulant). Tetramethyl Guanidine was diluted from vapor headspace to 2 ppm and was detected with the N5's existing SPH, non-optimized, sensor array (FIG. 8). Similarly, in FIG. 9, the acetamide was detected using the existing SPH sensor array to show feasibility. Thus, we can support the feasibility of acetamide as the basis for SPH sensing mechanisms. This can be used as a probe molecule for sensor design and optimization efforts. This also has the potential to discriminate PBA from NTAs, CWAs, and TICs. Note too, the responses to the two simulants, even without sensor array optimization for NTAs and PBAs, point to the potential for discrimination of these two different threat types from one another. FIG. 10 illustrates further potential simulants.

### Evaluation of Current Aerosol Samplers

An assessment of the current state of the art for sampling and transferring chemical aerosols, whether presented as liquid droplets or solid particles, was conducted. In addition, research was also conducted for the detection of Biological Warfare Agents and bacteria and viruses, in general, as they will present as liquid or solid aerosols depending on the manner of dissemination. A summary table of the key parameters for the major types of aerosol collection was compiled as shown in Table 2.

TABLE 2
Summary of aerosol collector approaches and key parameters

| | | | | | Thermo- |
|---|---|---|---|---|---|
| | Inertial onto | Inertial | | Filter | phoretic |
| Parameters | solid substrate | into liquid | Electrostatic | Collection | samplers |
| Particle | Bio and chemical | Bioaerosols | Combustion | All | Combustion |
| type/size, | aerosols | (1-5 um) | UPM (<0.5 | | UPM |
| um | (0.5-10 um) | | um) | | (<0.5 um) |
| Analysis | Optical in-situ, | Bioassay, | Filtration, | Analytical | Optical |
| type | micro-fluidic, | microfluidic | optical, and | Chemistry | |
| | desorption ex- | assay, | real- time | (extraction, | |
| | situ | continuous | sizing with | digestion) | |
| | | monitoring | DMA | | |
| Scalability | Good | Bulky | Expensive | Large | Bulky |
| (microscale) | | | to | eluent | |
| | | | miniaturize | sample | |
| Integration | Need for micro | Complex liquid | High voltage, | Sample | Thermal control, |
| Challenges | components, | handling, | long pass | prep, high | low efficiency, |
| | PM >0.5 um | large volume | length, limited | effluent | limited to UPM |
| | | | to UPM | volumes | |

The aerosol collection challenges include the need to detect particle sizes in the respirable range, roughly 1-10 micron range, and finger particles in the 0.1-1 micron range. It was recognized that some of the threat agents can present as even larger particles (e.g., >10 microns). For the larger particles, the particle settling rates tend to be high, and therefore the concentration could be quite low depending on the distance from the dissemination device. It was also recognized that there will be a host of non-target airborne particles, such as atmospheric dust re-suspended during natural meteorological or manmade events and environmental pollutants such as smoke and diesel. In addition, though there are several commercial devices for aerosol collection, an approach is needed that will be consistent with a compact, low SWaP, person-worn device.

For the ChemBadgER, it is desirable to have a collection and concentration approach that utilizes the aerodynamic properties of the aerosol particle (or drop) without additional mechanisms to control particle trajectory. Impaction is a common approach that should be scalable for the present application. Here the aerosol would be collected onto a solid-state material or substrate of some sort (e.g., a filter, a wire mesh, a porous material) followed by desorption to the vapor state and transfer to the SPH sensors. However, the smaller, ultrafine particulate matter (UPM), typically defined as particles <0.5 μm, will typically follow gas streamlines, and collection via impaction would not result in a high capture efficiency. For the ultrafine aerosols, an electrostatically assisted approach, that is, imparting a charge to the particles, could be beneficial. Electrostatic methods also are good for lower flow velocities that would likely be in effect for a small, wearable device.

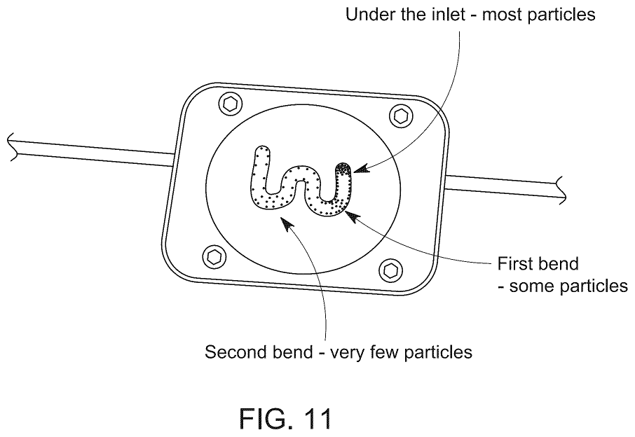

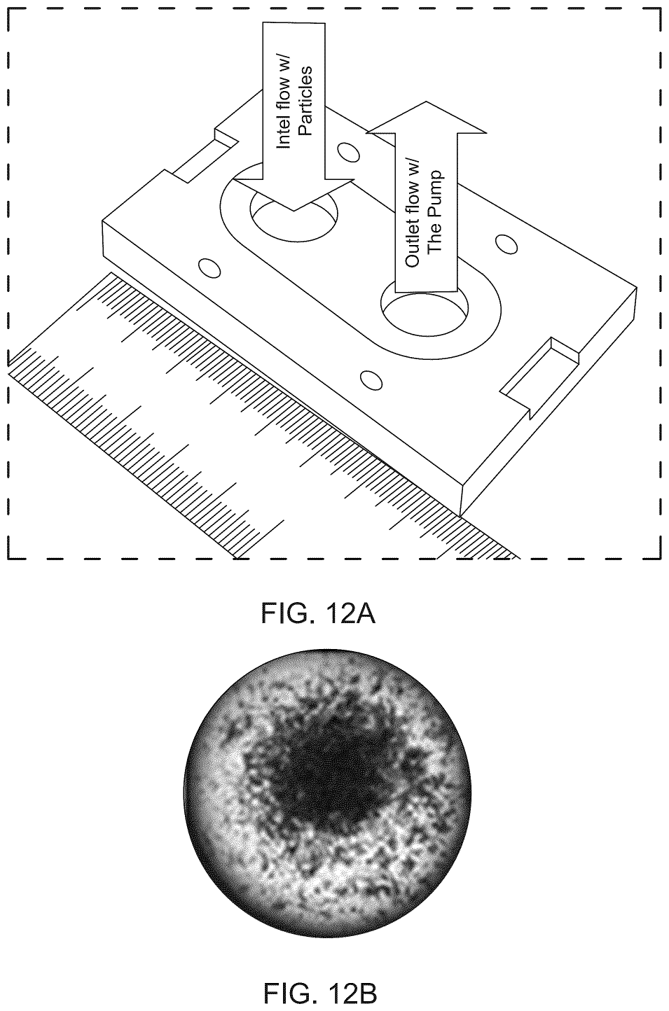

Two examples of aerosol impaction collector designs are shown in FIGS. 11 and 12A-12B. FIG. 11 uses a “W” microchannel design and FIGS. 12A-12B uses a microwell design. Both designs have been prototyped and demonstrated for aerosol collection, but require modification for use with the SPH sensor array. These impaction designs also feature a small footprint suitable for sensor integration and offer orientation independent operation. For FIG. 11 (the “W-design”), capture efficiency (CE) studies were conducted and show good CE for 2-micron particles in the 70%-95% range and at flow rates consistent with the SPH sensor design. The FIG. 11 design uses 2 μm fluorescent polystyrene spheres at 3 slpm flow rate. Microparticles in FIG. 11 can be collected with a small volume. An electrostatically assisted aerosol collector can also be used. Further, designs for a non-ASIC initial prototype were also evaluated for the ChemBadgER.

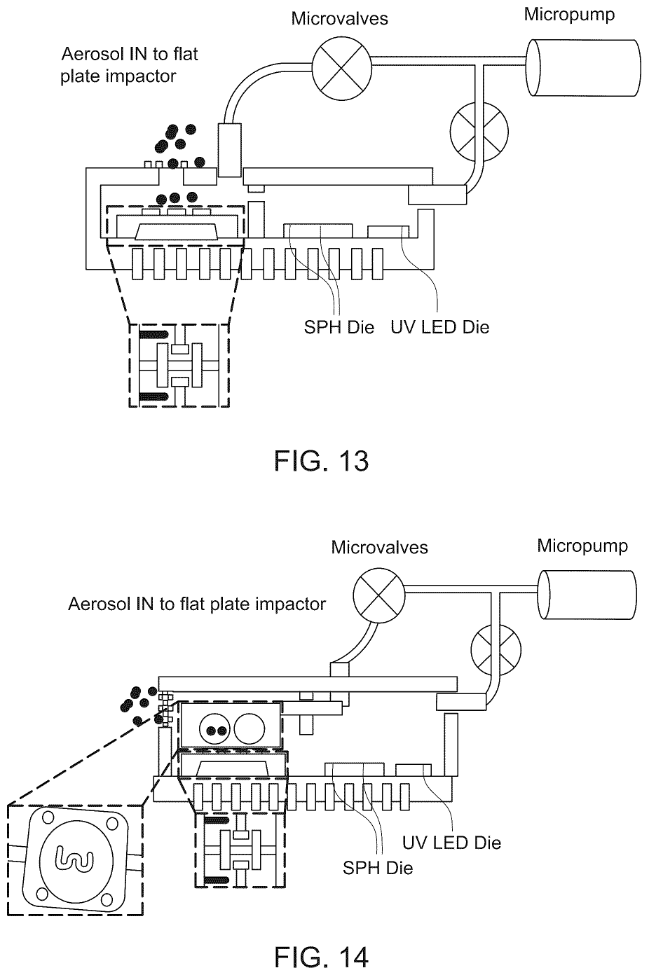

An embodiment of an integration of a micro-aerosol collector with the SPH sensors is shown in FIG. 13. In FIG. 13, a microheater is used at the base of a microwell. Aerosols are collected in the microwell by impaction and then the sample is desorbed using the microheater for transfer to the SPH sensor die. The microvalve is 20×8×5 mm in size. The micropump is 20×20 mm and 1.85 mm thick in size. However, the micropump is generally 4×1 cm or smaller depending on pumping power. There are a couple of different microheaters that can be used. One microheater is 5×5 mm in size. Another microheater is 3.9×5.2 mm in size. However, a microheater die can be as small as 1 mm.

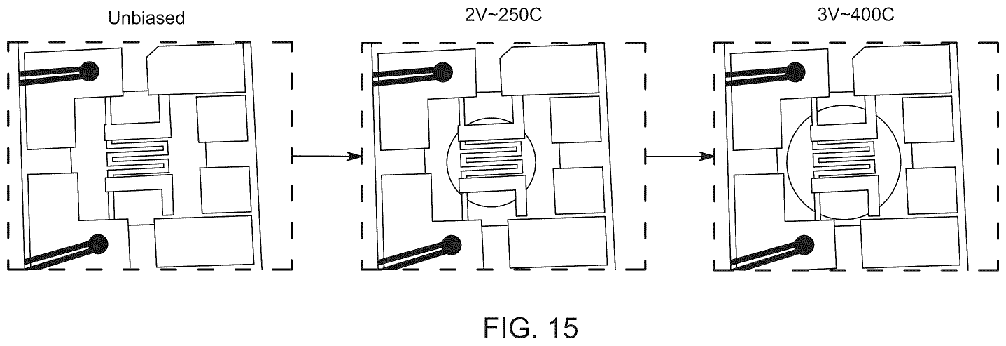

Another embodiment of an integration of a micro-aerosol collector with the SPH sensors is shown in FIG. 14. This embodiment is also based on impaction like FIG. 13, but uses a microfluidic channel design for aerosol capture. Again, a microheater is used to desorb the sample to the vapor state and for transfer and detection using the SPH array. The microvalve is 20×8×5 mm in size. The micropump is 20×20 mm and 1.85 mm thick in size. However, the micropump is generally 4×1 cm or smaller depending on pumping power. There are a couple of different microheaters that can be used. One microheater is 5×5 mm in size. Another microheater is 3.9×5.2 mm in size. A microheater device is illustrated in FIG. 15. The microheater is less than 1 mm in size, with the capture area on the microheater being 250 microns. The device was tested for the ability to rapidly heat and cool the device over the extended temperature range that might be required to desorb a wide range of chemical threat types. A test was also conducted to show that malathion, a low-volatility organic compound and a CWA simulant (vapor pressure 1.78×10-4 Pa), could be rapidly vaporized using the microheater device. For this test, malathion was deposited onto the microheater, the heater power was ramped and the chemical was rapidly flashed from the heater surface. −0.8 mg of 50 wt % malathion was evaporated from the device surface.

### Breadboard Development: Aerosol Capture Device

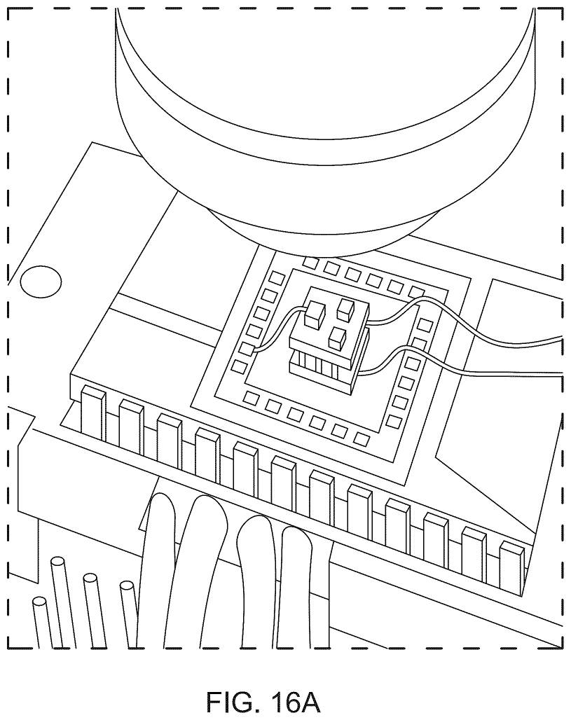

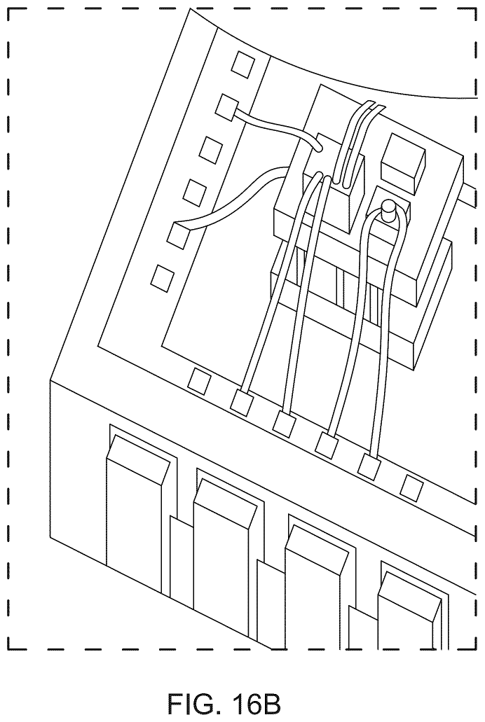

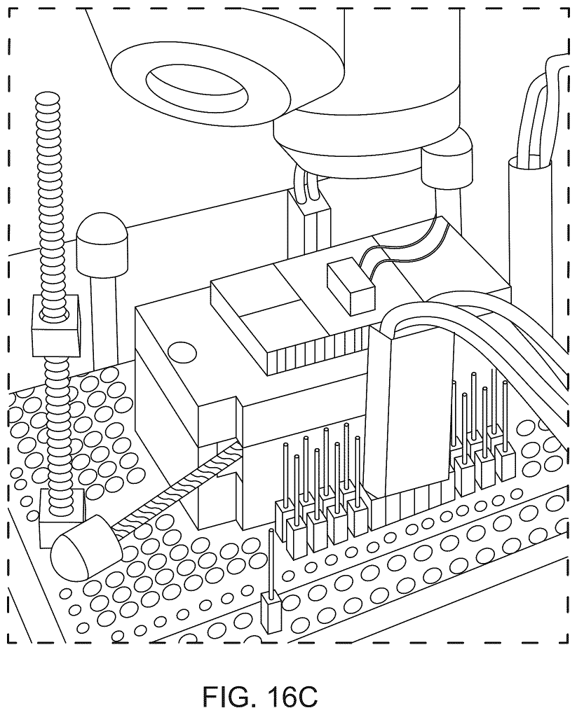

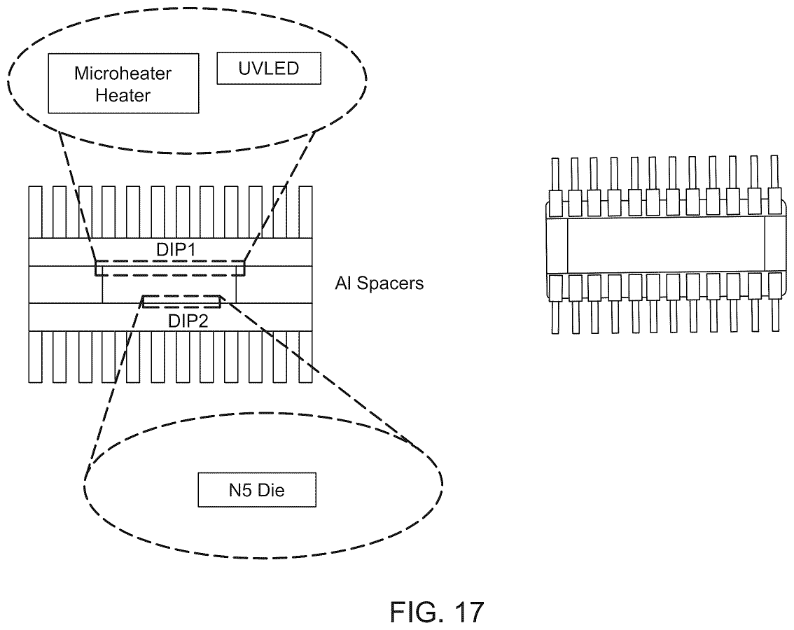

A breadboard test system was developed as shown in FIGS. 16A-16C. This included a microheater and a Peltier cooling device, integrated with the SPH sensor die. In the figure, it can be seen that a microscope is used to visualize the microheater and SPH sensor die due to the miniature size of the components. FIG. 16A illustrates the SPH sensor die and microheater device arranged onto surface of the Peltier Element. FIGS. 16B and 16C illustrate the aerosol sensing configuration placed onto a pinout board for the Peltier/microheater input control and SPH sensor die signal. The microheater/SPH sensor die component is shown in FIG. 17. The SPH sensor die and microheater device are each about 1 mm by 1 mm. Once the microheater detects an aerosol and processes the information, the microheater can be reused and the substrate cleaned by applying a voltage of 3 volts for less than 5 minutes. This ensures that the sensing device continues to operate in real-time for rapidly changing conditions. Further, this ensures a low cost because it does not have to be replaced after each use. The breadboard device was used to demonstrate the feasibility of desorbing a simulant and then detection of that vapor using the SPH sensor array.

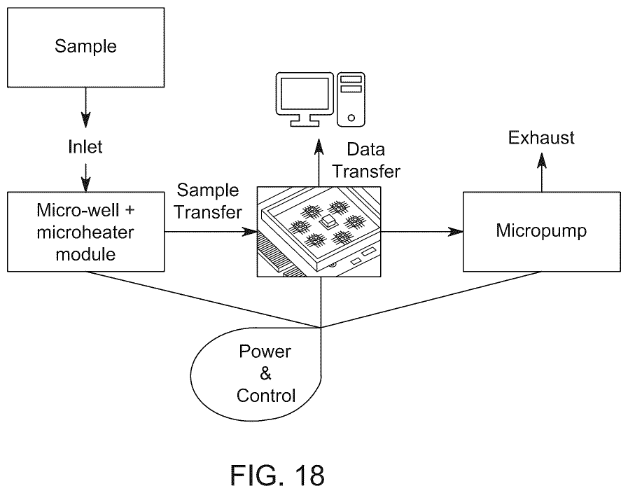

FIG. 18 illustrates a method of the breadboard testing using the microwell aerosol collector. A sample is collected by the microwell inlet and is heated by the microheater, and then transferred to the SPH array, which is then analyzed by the computing component. The sample proceeds to the micropump and exits the device via the exhaust.

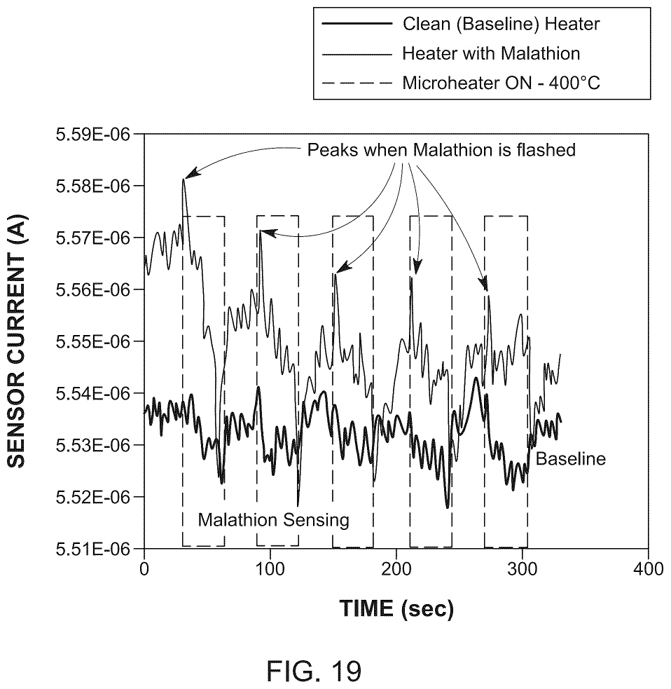

In this test, the microheater/sensor die unit was either spiked with malathion or left blank. FIG. 19 shows the detection of malathion: when the temperature is ramped, the chemical is rapidly desorbed and the vapor is detected by the SPH sensor array. When the same experiment is performed without malathion there is no response from the sensor array using the same protocol as with the simulant. The total mass of vaporized malathion was 0.2 mg of 50 wt %.

The testing demonstrated the breadboard implementation of the modules and the end-to-end aerosol detection system including: aerosol sampling, aerosol capture, aerosol-to-vapor conversion, vapor delivery, and sensing module.

The performance for the Person-Worn Chemical Aerosol Detection is shown in Table 3.

TABLE 3
Summary of ChemBadgER Performance

| | ChemBadge | ChemBadgER |
|---|---|---|
| | (Vapor) | (Vapor + Aerosol) |
| Limit of | Nominally 100 ppb with | 10 ppb for |
| Detection/ | analyte—specific | aerosolized threats |
| Sensitivity | variations | |
| Selectivity | TICs/CWAs/ | TICs/CWAs/ |
| | Environmental Gases | NTAs/PBAs/ |
| | | Environmental Gases |
| Threats Detected | Vapor | Vapor + Aerosol |
| Time to detection | <30 s | <60 s |
| Power | Up to 20 hours of | 12 hours battery life |
| consumption/ | battery life | |
| Duration of | | |
| Operation | | |
| SWaP | 3.5″ × 2.5″ × 1.2″, <1 lb | 5″ × 3″ × 2″, 1 lb |
| Consumables | Filters (1 yr), | Filters (6 months), |
| | Sensors (3 yrs) | Aerosol Collector (1 year) |
| | | Sensors (3 yrs) |
| Communication | USB and Bluetooth | USB and Bluetooth |
| | Low Energy | Low Energy |

### Sensor Array Development for Detection of Target Threats

N5 has developed a sensor array for the detection of CWAs, NTAs, PBAs, and relevant degradation products (to the extent these are known). Sensors for additional TICs can also be added, if needed. N5 continues to assess potential differences in pharmaceutical grade and illicitly manufactured PBAs. The selection process for the materials library includes the identification of potential simulants for fentanyl, nitazenes, and NTAs, and the development of plausible reaction pathways for interaction with the SPH sensors (e.g., FIG. 7). The sensor materials library will be used in conjunction with metal co-catalysts to impart sensor signature diversity with the multi-element array. N5 can utilize its existing materials science toolbox to design an initial array of ChemBadgER SPH sensors as shown in Table 4.

TABLE 4
Chemical Agents, Simulants and Degradation Products

| | Target | | Degradation Products |
|---|---|---|---|
| | Chemical | Simulant | of Target Chemical |
| PBA | Fentanyl | 1-benzyl-2- | Pyridine, Styrene, Benzaldehyde, |
| | | piperidone | Aniline, Phenylacetaldehyde, N- |
| | | | Phenylpropionamide, Methylpyridine |
| | Nitazene | nitrobenzene | o-, m-, p-Nitrophenol, |
| CWA | GB | | Nitrosobenzene, Phenol |
| | | | Diisopropyl Methyl |
| | | | Phosphonate (DIMP), |
| | | | Methylphosphonic Acid (MPA) |
| | VX | Malathion | Diisopropyl ethyl mercaptoamine, |
| | | | Ethyl methyl-phosphonic acid |
| | | | (EMPA), S-(2-Diisopropylaminoethyl) |
| | | | Methyl Phosphonothiote, |
| | | | Methylphosphonic Acid (MPA) |
| NTA | A-232 | Tetramethyl | A-232 Acid, Phosphoric Acid |
| | | Guanidine | |

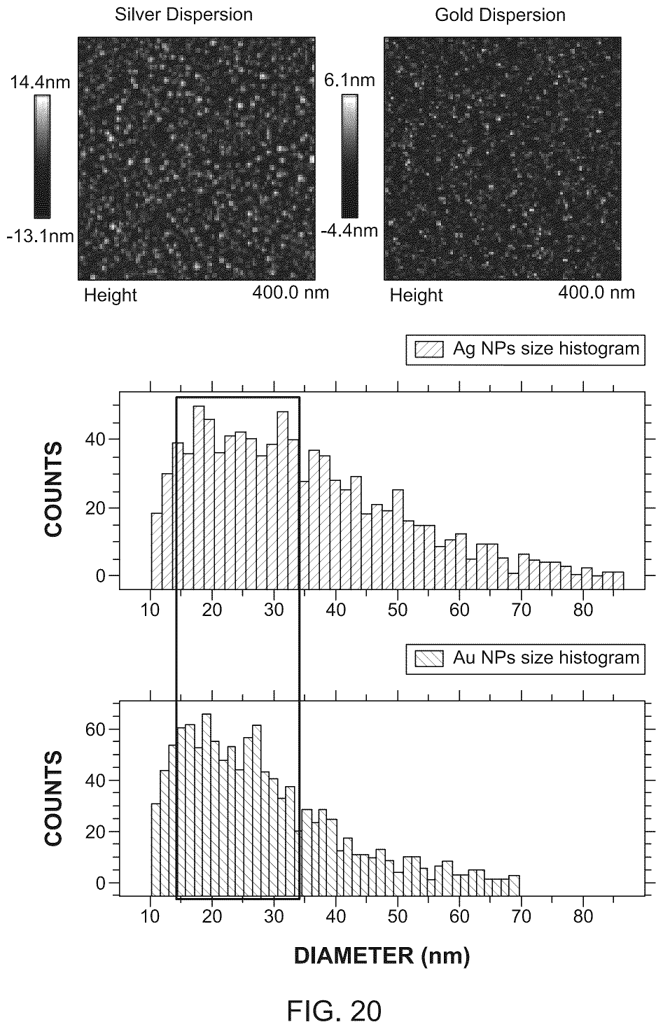

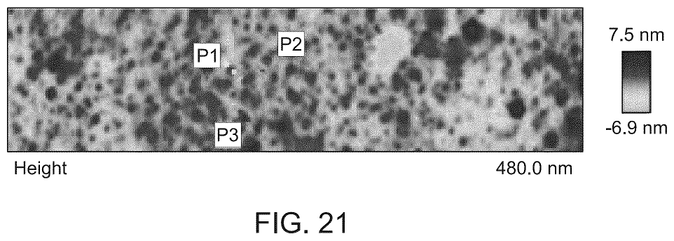

As discussed above, N5's SPH array uses sensors for TICs, CWAs, and environmental gases. However, the SPH array can be integrated with further components to detect NTAs and PBAs. The sensor design is approached on two fronts. The sensor architecture addresses the optimization of physical parameters including doping, thickness, width, length, and orientation. These are evaluated for the impact they have on power consumption, signal-to-noise ratio, response magnitude, minimum detection limit, and range. The second element of sensor design is materials design: the selection of functional layers and co-catalysts. In the SPH array/aerosol integration embodiment, new sensors can be fabricated using the process presented earlier in FIG. 4. N5 has developed wafer-level control of uniformity of dispersion of functional materials. Referring to FIG. 4, the process entails: 1) GaN backbone etching, 2) deposition of ohmic contact, 3) deposition of functional layers (i.e., metal oxide semiconductor and metal cocatalyst, and 4) deposition of bond-pad. N5 has developed manufacturing capabilities that allow for precise control of the functional material dispersion on the wafer scale. N5 has an in-house EvoVac deposition system featuring dual-source deposition, magnetron sputtering, and electron beam evaporation capabilities. This is suitable for preparing wafers up to 8″. This industry-standard ultra-high vacuum deposition system enables tight control of particle size and layer thickness as shown in FIGS. 20 and 21. For example, FIG. 21 illustrates an electric field microscopy (AFM) for dispersion estimation and film thickness.

### Utilization of Metal Oxide Semiconductors and Metal Cocatalysts

The N5 sensor design is fundamentally different from conventional metal oxide sensors. Even though the functional layer is typically a metal oxide the SPH arrays benefit from the additive effect of many types of metal co-catalysts and the use of photocatalytic reactions. N5 has developed an extensive materials library used to design sensors for specific purposes. N5's material library will be screened and materials down selected for the ChemBadgER application. Utilizing a metal cocatalyst significantly enhances both the sensor responsivity and selectivity for the desired chemical analyte. Studies have shown that the functional metal significantly influences the adsorption energy of targeted analytes, and subsequently their sensor selectivity. The metal functionalization strategy is one of the most innovative and differentiating features of N5's SPH sensors. Because most of the current MOx sensors work at high temperatures (typically between 200 and 400° C.), it is difficult to apply this cocatalyst on the metal oxide layer due to concerns with sintering. On the other hand, the SPH sensors offer great design versatility, exploiting a wide range of material combinations and enabling purpose-designed, selective vapor sensors for specific applications.

N5's typical array testing board is capable of mounting twenty-six sensor test dies (with onboard humidity and temperature sensors). The vapor sensing performance is determined by measuring the resistance change of two sensor elements in each sensor die. As such, fifty-two sensor elements can be measured at one time.

The N5 SPH sensors have been developed over 10 years of research to overcome the limitations of chemiresistor transducers including non-linearity and poor stability which impact sensitivity. In addition, the SPH design has eliminated the need for operation at elevated temperatures. One of the key methods to lower the detection limit is to reduce the critical dimension (CD) of the sensor, much like microprocessors get faster due to a reduction in process nodes. Testing of two different sensor elements provided results where there was ten times improvement in the detection limit, which can be achieved by reducing the CD from 350 nm to 200 nm. In our past experiments with ultrathin wires, we were able to demonstrate 0.5 ppb level detection with explosives. N5 is also developing frequency domain measurements to improve detection limits, selectivity, and SNR.

### Aerosol Sampler

Aerosol collection and analysis for wearable sensors require a compact solution compatible with the SWaP requirements. The aerosol collection module has been designed, developed, and tested for operation with the SPH sensor array. The prototype performance was characterized initially in the laboratory using aerosolized chemical threat simulants. Many of the chemical, biological, radiological, nuclear, and explosives (CBRNE) targets can be disseminated, and represent a respiration hazard, in the 0.5-5 micron range. Particulates in this size range have been efficiently captured using inertial impact collection methods. Examples include: (1) single organism bacterial spores (0.5-1 micron) and their clusters (up to 5 microns) and droplets containing other biological targets, i.e., viruses; (2) solid and liquid chemical agents; and (3) atmospheric dust re-suspended during natural meteorological events and other signatures of interest absorbed on the particle. A miniature collector (for 0.5-5 micron particles) could be adapted for ChemBadgER use. The aerosol would be collected directly onto, or in close proximity to, a thermal desorption substrate at flow rates compatible with the SPH sensor array (approximately 1-3 liters per minute). Particle capture, followed by in situ desorption allows for rapid analysis as the vapors and introduction to the sensor array in near real-time.

The main challenge for collecting coarse (PM10) and fine (PM2.5) aerosols is the need for sampling at high flow rates because the concentration of fine and coarse particles in the atmosphere is typically low due to the high settling rates of such particles. For coarse and fine aerosols, efficient concentration and collection can be achieved by utilizing their aerodynamic properties without additional mechanisms for particle trajectory control. Aerosol collection strategies also require a compact solution that is compatible with the SWAP requirements for the small sensor platform. One approach is collecting particles on a solid-state substrate and in situ particle desorption to analyze the resultant vapors. Efficient collection of particulate matter to increase the particle concentration on the collection substrate can also enable alternative analysis methods, both in situ and ex situ, should that be desired. Given the wide range of chemical threats, in terms of chemical composition and vapor pressure, particular consideration will be given to the use of a staged thermal desorption process. This could provide one additional mechanism (in addition to the fundamental sensor design) for imparting selectivity as a function of vapor pressure for the adsorbed particles (liquid or solid aerosol).

### Design I—Microwell Impactor

As discussed above with respect to FIGS. 12A-12B, a microwell can be adapted and integrated with the SPH array. This approach consists of a microwell aerosol collector combined with an aerodynamic focusing (AF) inlet as shown in FIG. 12A. FIG. 12B illustrates the microwell (D 1.5 mm) with particles collected using personal exposure monitor in epidemiology study. The addition of the aerodynamic particle focusing before the aerosol exits the nozzle yields significant improvements in device performance. The primary advantage of the microwell design is that it can be implemented in a small size and has lower thermal mass. The combination of the AF inlet and the angle of the microwell wall enables trapping the aerosol inside the well structure. The microwell feature collects nearly all of the particles, regardless of their size in its submillimeter diameter region, while a flat impactor has a significant dispersion of the collected aerosol. The AF microwell collector can collect highly concentrated particle samples in a 1 mm in diameter collection site. Particles stay in the well because their bounce is redirected toward the center of the well, increasing the sample collection density. The collection efficiency of the microwell collector shows a smaller dependency on the particle bounce when compared to the flat impactor. The flat collector exhibits the reduced particle collection for the Stokes number, FStk>0.4, due to particle bounce. In addition to the particle Stokes number (or impaction velocity magnitude), the bounce characteristics depend on the particle and surface properties as well as the environmental conditions. Iterative Computational Fluid Dynamics (CFD) simulations will guide the design and optimization of the device.

### Design 2—Microchannel Collector

As discussed above with respect to FIG. 11, a microchannel can be adapted and integrated with the SPH array. A microchannel collector was designed, with the capability of automated operation and integrated with a microfluidic assay. The collection module utilizes a microchannel that receives the airflow and can be coupled to the liquid channel to elute collected particles. Features of this design relevant to the ChemBadgER development are: orientation-independent operation; desorbed vapor stays contained within the collector; and a small footprint for integration due to the planar geometry of a fluidic path. The microchannel collector for particles larger than 0.5 μm is based on a centrifugal impaction concept. Collector geometry has been optimized using computational fluid dynamics (CFD) to minimize pressure drop and increase collection efficiencies. Fluorescent polystyrene spheres were used to visualize particle collection inside the microchannel collector. FIG. 21 illustrates photographs of florescent particle collection regions. The air is aspirated by the air inlet on the opposite side (from imaging) on the top right of each collection channel. In a microchannel collector, 2 micron particles are collected near the inlet, while smaller particles with lower Stokes numbers are more evenly distributed in the channel.

The collection efficiency with the microchannel impactor is a function of particle size and the flow rate. The microchannel collector shows 50% collection efficiencies for 0.5 μm particles, and close to 100% efficiencies for particles larger than 2 μm. The advantages of the microchannel collector for collecting particles in the 0.5-10 μm size range are apparent for environmental monitoring and integration with small detectors. The design has been previously validated for flow rates of 1-2 slpm for bioaerosol capture.

### Aerosol Sampler Fabrication and Testing

The aerosol inlet and sampler design can be fabricated to optimize the aerosol collection module for operation with the SPH sensor array platform. The miniature aerosol collector for chemical aerosols (0.5-5 microns) can be adapted. The performance of the collection cartridge in the aerosol chamber using polydisperse aerosols can also be evaluated. Using medical nebulizers, the particles will be aerosolized in a custom 0.3 m³ well-mixed aerosol chamber. The aerosol concentration is spatially uniform with the operation of the mixing fans. An automated control module can hold the humidity in the sealed chamber range of 30%-90% RH. Real-time particle concentration and size distribution will be measured using SMPS (TSI Nanoscan model 3910, dₚ=10-300 nm) and aerodynamic particle sizer (TSI APS 3321, dₚ=0.3-20 μm), allowing the collector performance to be determined over a wide range of particle sizes. Electrostatically dissipative tubing can be used to avoid any electrostatic losses in the sampling train.

Two types of challenge aerosols can be used: (i) Arizona test dust (ATD) with aerodynamic particle diameter dₚ=0.5-10 μm and (ii) NaCl particle size can be varied by the concentration of salt in the nebulizing solution (dₚ=10 nm−2 μm). Once aerosolized, the particle dynamics are not affected by their chemical composition. APS and SMPS can be used to measure the particle concentration in the reference and collector channels. The flow between two channels is switched using pinch valves. At least ten measurements are taken for each flow rate to calculate the collection efficiencies. Collection efficiencies can be determined using an aerodynamic particle sizer (APS) by alternating the flow between the microchannel and the reference sampling. The collection efficiency (CE) can be calculated with the two measured concentrations using Equation 1, where C_{ref}, C_{col} are concentrations with a given size particle in the reference channel and channel with the collector, respectively.

$$CE = \frac{{C}_{ref} - {C}_{col}}{{C}_{ref}}$$

The preset flow rate for the APS is 1 slpm and for SMPS is 0.8 slpm. The higher flow rate can be achieved by adjusting the compensation flow rate provided by a vacuum pump. The operation flow rates 1, 2, 3, and 5 slpm are set using a mass flow controller; the flow rate to APS and SMPS can be verified using a TSI 4420 flow meter that does not impede aerosol transmission. This approach can be used to quantify the performance of different aerosol devices and PM sensors and to determine textiles filtration efficiency.

Sensor Array Integration with the Collection/Desorption Module

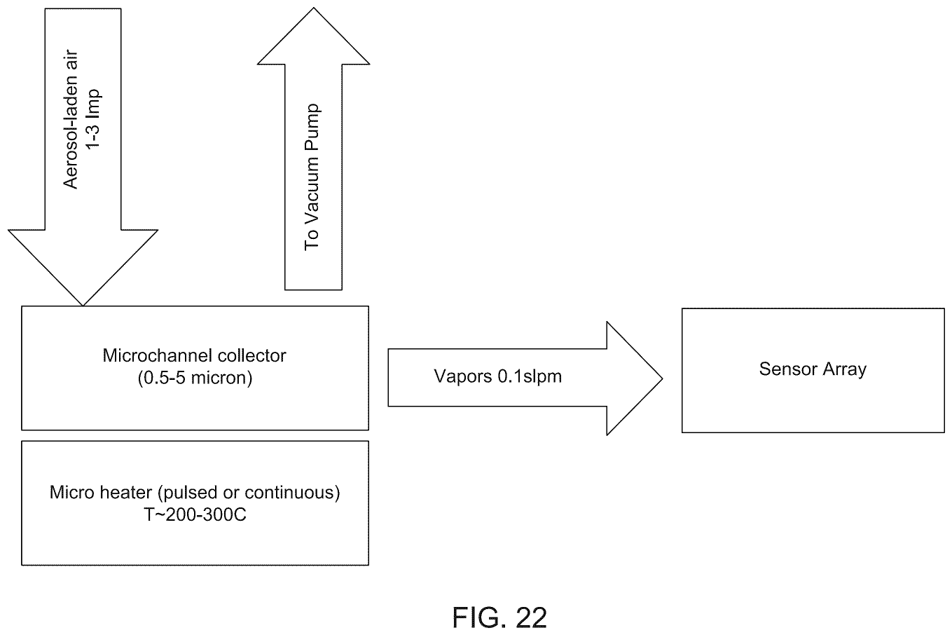

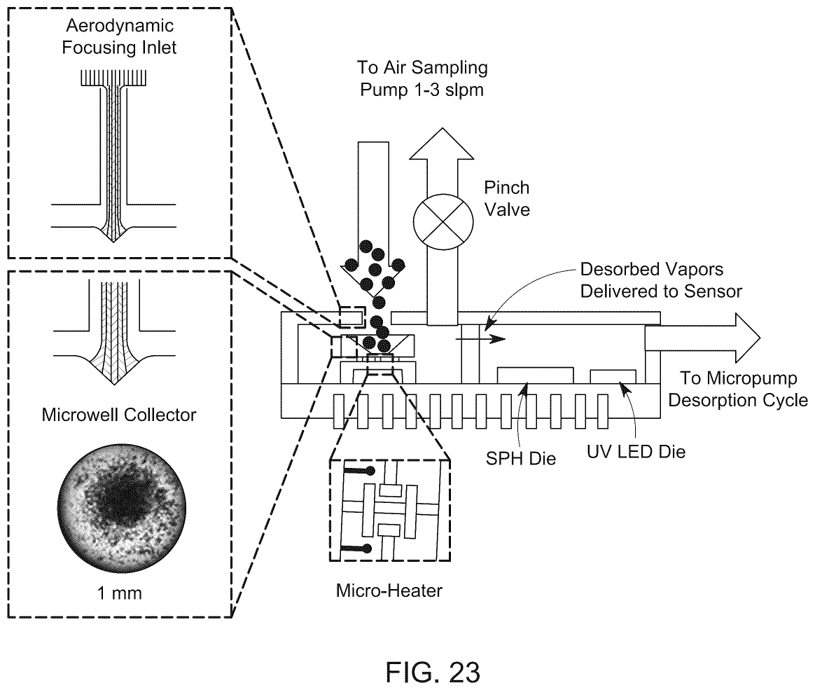

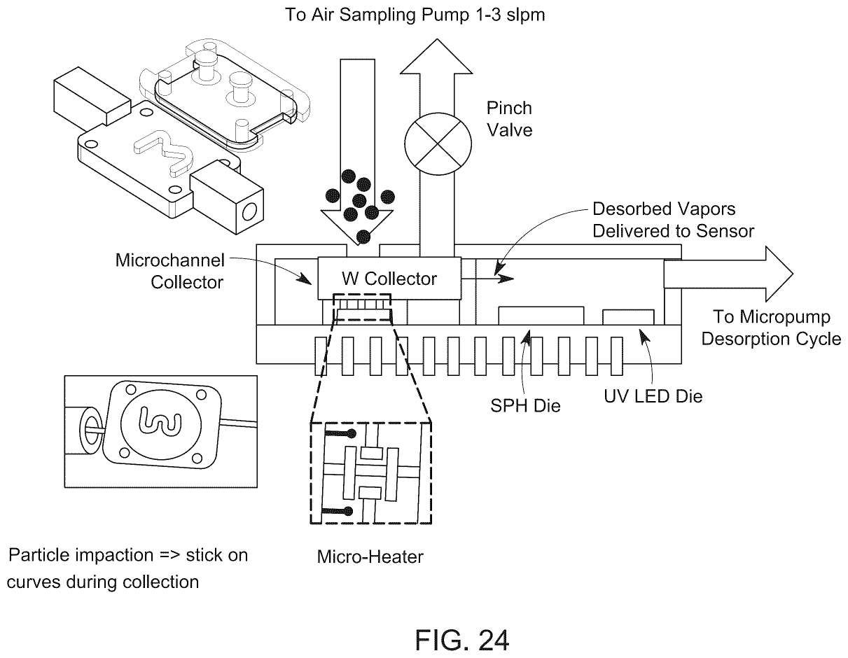

In an embodiment for the aerosol detector, it is important that the sampling and detection of liquid and solid aerosols is done in the respirable range (approximately 0.5-10 microns). Different design approaches for sample recovery and transfer were evaluated. As discussed above, using a microheater to desorb the collected sample via thermal vaporization can be used. FIG. 22 shows a block diagram of how these systems would be integrated with the sensors. FIGS. 23 and 24 shows in greater detail how the two designs are integrated with N5's SPH sensor array. In an embodiment, FIG. 23 is based on a microwell collector with an integrated microheater for collection and desorption (designated Design M). In another embodiment, FIG. 24 is based on the W-channel collector with a microheater outside the channel for aid in desorption (designated as Design W).

In FIG. 23, the aerodynamic focusing inlet via the air sampling pump brings in the particles to the microwell collector. The air sampling pump includes a pinch valve so that the flow can be controlled. The particles are then heated by the microheater and the desorbed particles are delivered to the sensor. The sensor includes an SPH die and a UV LED die. Once delivered to the sensor, the desorbed particles are analyzed in the desorption cycle.

The embodiment of FIG. 24 works similar to FIG. 24, but instead of the microwell collector (Design M), a W-channel collector is used (Design W).

The embodiments of FIG. 23 and FIG. 24 are two different aerosol captures approaches that can be integrated with the SPH sensor module. In both embodiments, the SPH sensor module can comprise a plurality of sensor dies, where each sensor die provides for a particular functionalization providing different receptor technology. For example, in the modified SPH sensor module for the embodiments of FIGS. 23 and 24, 12 sensor dies can be used to allow for 42 channels for functionalization of data. When adapting and integrating the aerosol collector with the sensor array, there are several parameters which are important considerations. Both embodiments of FIG. 23 and FIG. 24 feature easily replaceable particle collection cartridges. However, in other embodiments, the collection cartridges may be disposable/consumable.

One parameter is the collection area, where the size of the collection spot is minimized, which will reduce the dilution of the recovered sample and would deliver the highest concentration of the analyte to the detector.

Another parameter is sample recovery. Vaporized sample transport is likely to introduce significant vapor losses. This is addressed by heating the device in the vicinity of the desorber to keep the low vapor pressure sample from sticking to the surfaces and by keeping the pathlength as short as possible.

A further parameter is fouling and recovery. Methods for cleaning or re-generating the device have been investigated. The thermal gradient in the device is likely to cause thermophoresis and some condensation of the desorbed sample to the surfaces. This is especially a concern for low vapor pressure targets.

Another parameter is thermal mass and selectivity. Reducing the thermal mass of the collector allows the utilization of a staged desorption cycle. Rapid desorption ramp-up can be followed by holding the temperature at the desired level to target compounds in a specific vapor pressure range and avoid chemical breakdown of volatile species. The temperature will be increased for the next stage to target the lower vapor pressure compounds. This approach is beneficial given the complex environmental matrix under the anticipated operational scenarios.

A further parameter is materials selection. The materials of construction should be suitable for withstanding the temperature changes associated with thermal desorption, and with adequate chemical resistance. This will require the fabrication of the collector in materials such as stainless steel, aluminum, or SiO₂.

### Dual Vapor/Aerosol Sampling

In an embodiment, the SPH sensor array is capable of both vapor and aerosol sampling and detection. Table 5 describes various design directions for a dual-use aerosol and vapor detector and the functional tradeoffs of the different approaches. One embodiment uses a single sensor module for both aerosols and vapor detection, and another embodiment uses two sensor array modules, one for aerosol threats and the second for vapor threats.

TABLE 5
Design directions and tradeoffs for a dual-use Aerosol and Vapor Detector

| | Dual-Mode Aerosol/ | Dual-Mode Aerosol/ | Discrete |
|---|---|---|---|
| | Vapor-Separate | Vapor-Common Sensor | Aerosol |
| | Sensor Modules | Module and inlet | Detector |
| Description | A single detector, with | A single detector for vapor | A separate |
| | separate sensor arrays | and aerosols, with a common | detector with only |
| | optimized for vapor | sensor array sensing both | Aerosol |
| | sensing and aerosol | environmental vapors and | Sampling |
| | sensing respectively | captured aerosol vapors | |
| Sensor | Aerosols and Vapors: | Aerosols and Vapors: | Aerosols only: |
| Capabilities | TIC, CWAs, PBAs, | TICs, CWAS, | TICS, CWAs, |
| | NTAs, 0₂, LEL | PBAs, NTAs, 0₂, LEL | PBAs, NTAs |
| Battery Life | Lower due to 2 | Longer than a detector | Longest battery life |
| | separate sensor | with a separate vapor | |
| | modules | sensor module | |
| Consumables | May reduce the | Consumables may be | Fewer consumables |
| | consumable | higher due to sharing | due to being |
| | frequency | a common inlet | Aerosol only |
| Cost | Higher cost due to | Cost per unit may | Cost is lower, |
| | extra components | be lower | but the overall |
| | | | cost for Vapor + |
| | | | Aerosol is higher |
| | | | as 2 detectors are |
| | | | needed |
| SWaP | The size and weight of | Sharing sensor module | Size/Weight is |
| | the unit are larger | and inlet | lower, but the |
| | than the integrated | improves SWaP | overall burden |
| | module due to the | | on end user |
| | extra pump and | | for Vapor + |
| | sensor module. A | | Aerosol is |
| | larger battery | | higher as 2 |
| | may be necessary. | | detectors are |
| | | | needed |
| Development | Moderate. With | Complex. Requires | Simplest to develop, |
| Complexity | separate sensor | significant algorithm | with no integration |
| | modules and inlets, | development to | with existing |
| | the integration is | manage the 2 sensing | hardware. |
| | mainly physical | modes simultaneously. | |
| | interconnectivity | Thermal behavior must | |
| | and power/ | also be carefully | |
| | connectivity | managed | |

### Modified ChemBadge Design

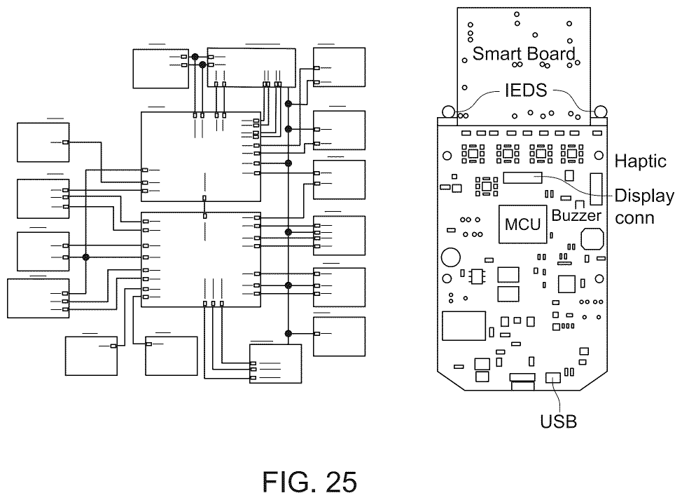

Integration of the aerosol sampler and sensors is based on proven ChemBadge electronics designs and hardware, as shown in FIG. 25. Existing schematics, components, and circuit boards designs from N5's ChemBadge vapor detectors can be leveraged, significantly reducing both development time and design risks. The key aspects of the modified design are integrating the aerosol sampling components including a microheater and sampling pump drivers. Additional SPH array sensors can also be added into the microcontroller and power modules.

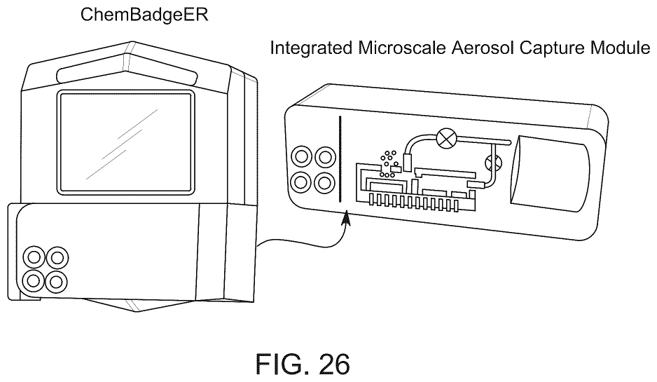

FIG. 26 illustrates an example of the ChemBadgER packaging with an integrated aerosol collector.

For the aerosol collector/SPH array, components are added for driving and controlling the additional pumps and heater components. The power consumption of the updated device will be benchmarked based on worst-case estimates. A new battery (rechargeable, lithium polymer) can be used to maintain a target time of 8 hours on a worst-case power budget, given device size targets of a wearable, handheld aerosol collection device. A solar component can be added in an embodiment, so that the battery can be recharged by the sun.

In an embodiment, an ASIC circuit design is used, which includes driving the microscale heating elements in the ASIC implementation. The ASIC circuit design is capable of implementing aerosol capture structures and electronic readout architectures to enhance portability, calibration, and throughput. The aerosol collector/SPH array design can be developed based on the current SPH ASIC design which has been tested. The current SPH ASIC design, discussed in N5's international patent publication noted above, includes an analog front end with calibration, analog to digital conversion, microprocessor, LED driver, low dropout voltage regulator, and I2C communication module. However, for the aerosol collector/SPH array design, it is necessary to eliminate instrumentation amp/opamp architecture to enable higher throughput for multi-analyte sensing, enabling quasi-digital readout to reduce power, incorporating low power strategies for the microprocessor, and incorporating constraints for heater elements (e.g., temperature, time).

In an embodiment, the IMAC requires a microheater driver. The voltage supply of the current microheater is well within current CMOS capabilities and a conservative estimate of 30 mA of current is expected. A PWM boost converter topology can be implemented and use power electronics layout techniques to ensure the robustness of the system. A potential challenge is a requirement for multiple pads (to connect to the outside world) to handle higher currents. Calibration can be provided using algorithms to account for the error and drift of the sensors. These can be implemented using a microprocessor embedded with the analog front-end (AFE) and readout electronics.

### Construction of Brassboard Prototypes

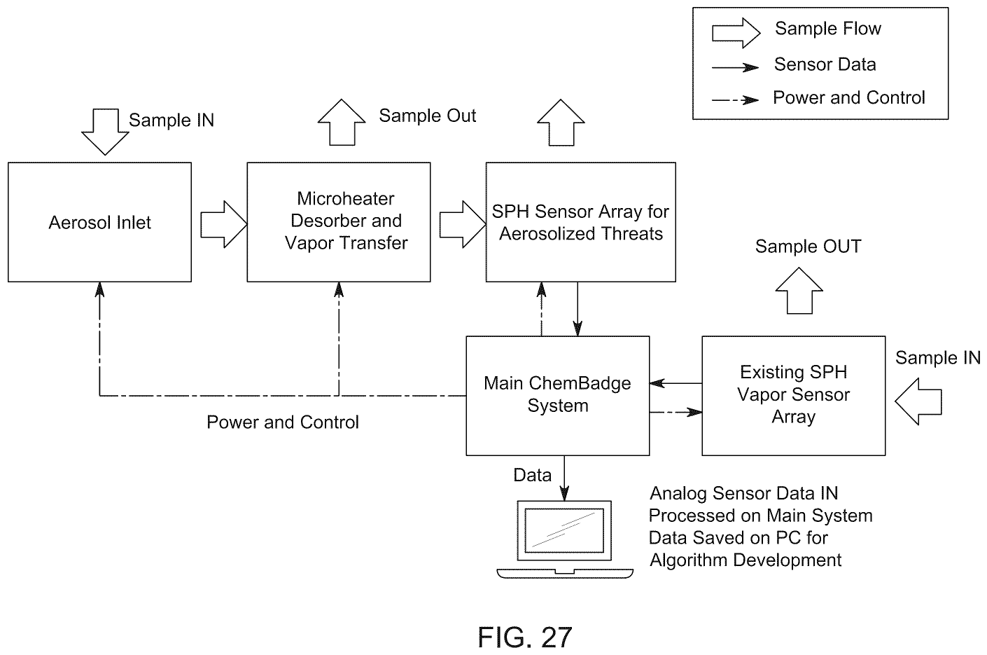

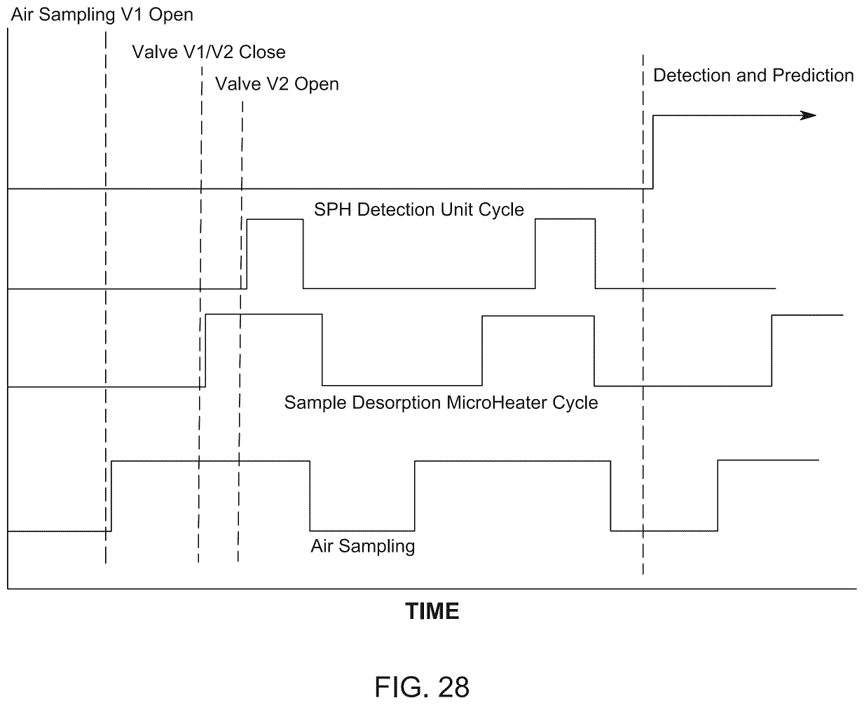

FIG. 27 illustrates another embodiment, which includes a block diagram of the full brassboard implementation to build a prototype of the aerosol collector/SPH array. The objective for this initial prototyping is the integration of all the components to test the functionality of the system and collect initial performance data. Existing designs and prototypes of N5's ChemBadge system can be leveraged to reduce the timeline for fabrication. Existing ChemBadge firmware can be modified to include additional functionality necessary to control the aerosol sampler inlet. This includes the sampling and desorption routines including optimizing the sampling time and power consumption. The testing of the prototype also allows for the development of an appropriate sampling sequence. This involves the control of the aerosol sampling pump, microheater driver, valves, and desorption pump to the sensor array as shown notionally in FIG. 28. The sequence, ramp rates, and sampling times can be studied and optimized to ensure successful aerosol delivery. This can be done by systematically testing individual subsystem performance in the development cycle to ensure each component is functioning as intended. Data collected on the Brassboard prototype can be used to build datasets for algorithm development and refinement in subsequent tasks. This will include test data of both vapor and aerosol simulants as described below. As data is collected on the subsystems and integrated prototype, correlations can be developed between vapor and simulant response data for CWAs, NTAs, and PBAs, so that a larger dataset is generated by combining data sources from both phases, thereby also aiding algorithm development.

The Brassboard and subsystems can be tested with aerosol and vapor samples at N5's facility. N5 can conduct full-system testing against aerosolized simulants and non-target chemical aerosols. This can be used to validate the full system functionality, and provide a baseline for system performance against the target specifications. Additionally, individual sub-systems can be tested so that performance of the fully integrated system can be compared against individual module performance, and any areas with performance loss or inefficiencies identified. An aerosol test chamber suitable for generating aerosolized chemical simulants and non-target compounds (interferents such as mannitol for the PBAs) can be used, which will leverage commercial off-the-shelf nebulizers (concentric Meinhard nebulizers for liquids, for instance), with a dilution and mixing stage before delivery to a test platform. A particle counter can be co-located in situ with the device under test.

Simulants can be used to test the functionality of the sampler and sensor array subsystems, and the integrated system, to establish initial detection performance benchmarks. Additionally, the data collected can be used to further develop and refine algorithms for aerosol detection and threat classification. Initial data can also be collected on environmental performance. This will be gathered by running the Brassboard units over time in indoor laboratory environments and limited testing in outdoor environments. This can be used to gain initial assessment data on the impact of environmental aerosols on system false alarm rate and consumables lifetime. Brassboard testing involves testing each subsystem (e.g., sensor array, aerosol sampler, heater desorber), and then testing the integrated system.

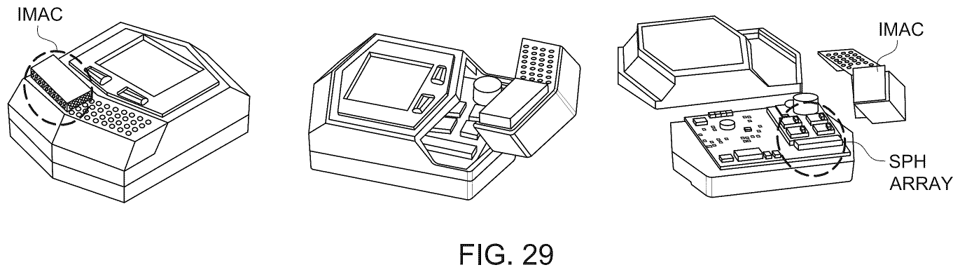

Another example of an embodiment of the ChemBadgER device, is shown in FIG. 29, which include the desorber electronics, the SPH sensor array, and the main electronics. The layout will be optimized on the whole system to reduce the size to a wearable form factor of 500 cm³ or less. As part of this, the power consumption of the device can be measured and improvements to hardware or firmware made. In particular, the SPH sensor array for chemical aerosol detection can be optimized in size and power by using test data to finalize the array size and improve the layout and packaging of the array. N5 has previously utilized Chip-on-Board (CoB) technology to integrate multiple dies near a circuit board and co-locate with LEDs and other components. By minimizing the size of the sensor module, less power will be required for any heating operations.

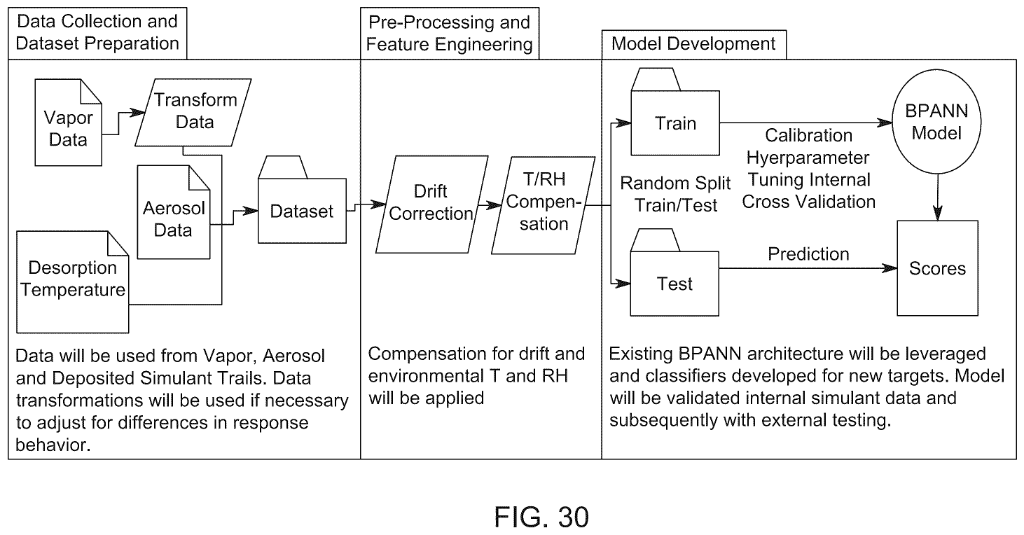

New data collected for simulants and target threats can be used to modify N5's chemical detection and classification algorithms for an expanded threat list including PBAs and NTAs. N5 has previously developed and implemented a Back Propagation Artificial Network for the classification of gas threats. This algorithm has already been successfully tested by N5 for threat classification on TIC threats. These algorithms can be leveraged and updated for the target threats of interest, such as PBAs and NTAs. The algorithm development process is represented in FIG. 30.

In summary, new data can be collected, both for aerosol and vapor simulants of PBA and NTA threats. Data processing can be used to transform data to account for response differences between vapor and aerosol sampling. Previous data processing approaches by N5 can be extended to the new data set and the classification model can be developed based on the existing BPANN architecture. In addition, N5 can evaluate the use of a staged thermal desorption sequence to add an additional dimension to the dataset. This can be based on the likely wide range of desorption temperatures and related parameters due to the diverse nature of the chemical threats (e.g., chemical composition, vapor pressure). This approach can impart an additional mechanism (in addition to the fundamental sensor design) for increasing selectivity as a function of vapor pressure for the adsorbed particles (liquid or solid aerosol).

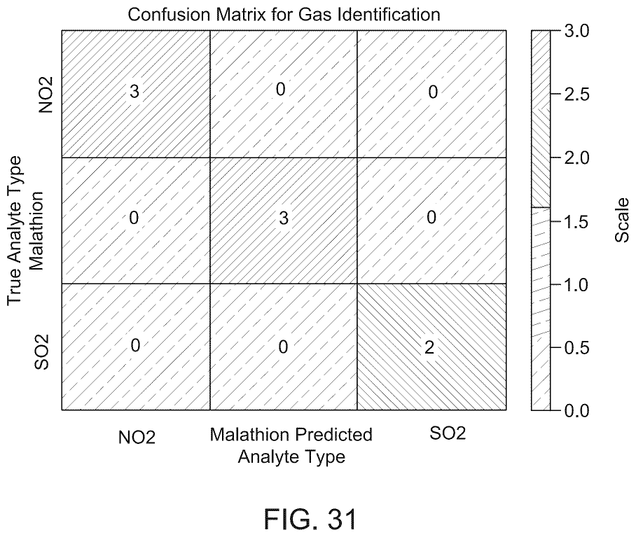

As a proof-of-concept, CWA simulant malathion and data from common environmental TICs SO2 and NO2 were used to train a BPANN classifier. From this laboratory-based dataset, the classification accuracy was 100%. The classification accuracy is typically represented by a confusion matrix for gas identification that compares actual to predicted classes, as shown in FIG. 31. By leveraging a sensor array with different sensitivity profiles to target gases, high classification accuracies are possible. To mitigate the impact of environmental factors on sensor response algorithmic approaches currently under development by N5 for TIC and CWA vapor detectors can be applied.

Low pass filtering to mitigate signal noise followed by signal correction via multi-variate regression can also be applied. Historical data for sensors exposed to various environmental conditions can be used as training data to extract coefficients for independent variables (Temperature and Relative Humidity). Environmental compensation algorithms can mitigate humidity and temperature impact on the sensor signal. Additional filtering of RH variables can be used to mimic sensor adsorption rates. With calculated coefficients for statistically significant independent variables, the sensor signal is corrected by subtracting the impact of each independent variable per unit of change. Signal correction for the effects of temperature and relative humidity based on the calculated coefficient from collected environmental data can be performed. In each case, the resulting corrected signal is flatter than the raw signal. These algorithms will can to be improved as more data is gathered specifically on environmental impacts on the NTA and PBA sensor arrays.

An updated housing for the aerosol sampling and desorption subsystem can be built and integrated into the ChemBadgER. Mechanical enclosure parts can be 3-D printed in durable resins and chemically resistant resins (such as Nylon). Previously, N5 has successfully fabricated detectors using 3D printing which has been suitable for field exercises and chemical testing. The updated designs of the Aerosol Collection Inlet, SPH sensor array and System Electronics can be fabricated and integrated into the housing. Prototypes will be assembled at N5 Sensors. N5 designs systems in a modular fashion to the greatest extent possible for adaptation or integration into other commercially off-the-shelf software (COTS) and developmental systems as needed. N5 has established critical fabrication partners over the years of prototype and product development. This has ensured on-time delivery of multiple prototypes to Government customers including the DHS and DoD.

Device firmware can be written, including menus for adjusting system parameters as needed. Manuals and training materials are be prepared that describe the system features, device operation, and maintenance and troubleshooting procedures. Training documentation includes both end-user manuals, as well as guidance for scientists/engineers for potential future independent evaluations of the device. Interface software for the prototype detectors is also developed for data download. N5 can leverage existing software and communication capabilities of its ChemBadge platform to allow operators to transfer data to reach back laboratories for further analysis. The software will enable the download of log files including raw sensor data, system configuration, and algorithm outputs. Currently, approximately one month of data can be stored on the detector. Depending on the number of new sensors and parameters introduced from the inclusion of the vapor sensor module, it is estimated that the new prototypes will be able to store approximately 10-14 days of data without significant modifications to the existing architectures.

### Live Threat Testing and Algorithm Refinement

Testing prototypes against a live PBA aerosol threat, such as fentanyl, can be performed. The exact specifications of the test conditions including threat, concentration, and any confounders will be determined based on the needed applications of the aerosol collector sensing device. Table 6 presents a test scenario with fentanyl and a cutting agent, mannitol.

TABLE 6
Nominal Test Parameters

| Target | Con- | Concen- | Particle | | Number of |
|---|---|---|---|---|---|
| Threat | founders | tration | Size | Environment | exposures |
| Fentanyl | Mannitol, | 1 mg/ | Distribution | Ambient | 3 neat and |
| | Lactose | m³ | centered at | Laboratory | 3 with |
| | | | 2 μm | (nominally | confounders |
| | | | | 23 C. and | |
| | | | | 50% RH) | |

For testing and evaluating the aerosol chambers for threat materials, the generation of aerosols is done using an industry-standard Vilnius Aerosol Generator (VAG). Filter samples can be collected and weighed to determine the total aerosolized concentration in the exposure chamber. Based on the results, VAG output settings and dilution ratios can be adjusted to achieve target concentrations.

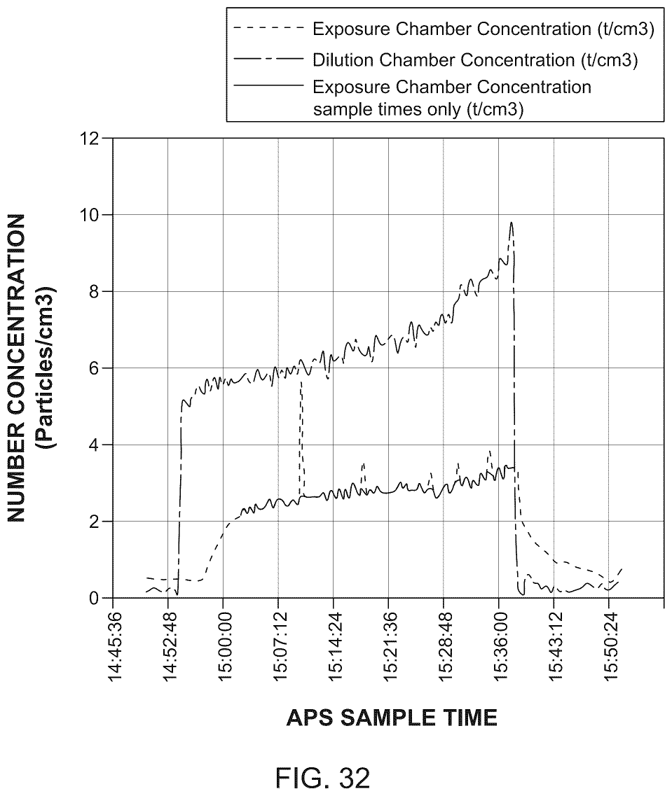

Uniformity of mixing and particle distribution in the chamber can be modeled using Computational Fluid Dynamics (CFD). Flow lines can indicate acceptable mixing/distribution of aerosol in the upper areas of the chamber. Particle injection results can be exhibited to show the homogeneity and mixing in the chamber. Also, in testing there were no consistent “hot spots” for mass flux, as areas of high and low concentration appear to be random and changing with time. This indicated acceptable mixing and particle distribution across the horizontal plane of the chamber. A homogeneity test was performed to validate the exposure chamber by aerosolizing 1 μm size-standard polystyrene latex beads (PSLs) and sampling with an APS at six locations. A plot of the aerosol concentration vs time in the dilution chamber and the exposure chamber is shown in FIG. 32. Based on the results of the live threat testing, modifications to the algorithm can be made if necessary, including transformations on the simulant training data to match the live threat response. Regardless of the threat tested, a test sequence can be performed with two separate exposure trials and approximately 1 day of N5 ChemBadgER “tuning” in between trials. During the “tuning” algorithm and sampling, sequences can be updated based on initial results from the first test to improve performance on the subsequent test.

### Fabrication, Testing, and Delivery of ChemBadgER Prototypes

Testing of the prototype ChemBadgER (aerosol collector/SPH array) detectors can be performed, where the testing is used to inform on key system parameters such as the detection range, response time, environmental performance, and lifetime. A test chamber for testing a fully assembled prototype with Temperature, Relative Humidity control can be used. This setup can be modified to include aerosol injection. The prototype testing before delivery is captured in Table 7. Recommendations for future development will also be outlined, with corresponding trade space analysis of any improvements or changes in the design.

TABLE 7
Final Prototype Testing Summary

| Test | Objective | Procedure |
|---|---|---|
| Operational | Informs | Collect background aerosols with |
| Environ- | MTBF | ChemBadgER prototypes in different |
| mental | and | environments, indoor and outdoor |
| Testing | MTBFA | conditions. Units are periodically |
| | | inspected for contamination and |
| | | clogging of inlets. Data is recorded, |
| | | and False Alarm Rate reported. |
| Chemical | Determine | Challenge detector against target |
| Aerosol | Sensitivity, | aerosols and simulants in a laboratory test |
| Response | Response | chamber. Conduct range finding trials to |
| Testing | and | baseline the detection limit of the system. |
| | Recovery | At a minimum, testing would include |
| | Time | stimulant chemicals from CWA, PBA, and |
| | | NTAs. The PBA simulant response will |
| | | be compared to the Live Threat Results. |
| Vapor | Determine | The detector will be exposed to vapor |
| Response | Vapor | including a subset of TICs, LEL and 02 |
| Validation | Sensing | conditions. This will be used to |
| | Performance | validate that vapor sensing performance |
| | of Dual- | is similar to vapor-only detectors. At |
| | Mode | minimum 2 TICs and LEL chemicals will |
| | Detector | be tested at IDLH and 25% LEL levels. |
| Sensor | Validate | Utilizing target aerosols or simulants |
| Repeat- | repeatability | conduct several replicate trials at |
| ability | of detection | concentrations approximately 2x the |
| Testing | | detection limit of the sensor. The |
| | | repeatability of the detector will be |
| | | studied, both alarm output and raw |
| | | signal analysis. |
| Interferent | Inform | Expose detector to aerosols of interferent |
| Aerosol | selectivity | or non-target chemical aerosols which |
| Testing | of system | include aerosols of compounds such as |
| | | lactose, mannitol, isopropanol, mineral |
| | | oil or other compounds. N5 will |
| | | develop the list of interferent species to |
| | | test having chemical properties (reactive |
| | | groups), physical properties (vapor |
| | | pressure) of operational relevance. |
| Environ- | Characterize | Utilizing environmental chambers, run |
| mental | system | temperature and humidity cycling routines |
| Testing | response to | to expose the Detector to different |
| | temperature | environmental conditions. Tests will |
| | and | include multiple data points in the range |
| | humidity | of 0-50 C. and 10-90% RH including |
| | | absolute humidity of 32 g/m³ |
| Power | Inform | Power consumption of the system will be |
| Con- | SWaP | measured during various operational |
| sumption | analysis | modes—Sampling, Sensing, Sleep, or |
| | and Battery | other modes. The battery life will be |
| | Life | measured in tests at various environmental |
| | | conditions (i.e., different temperatures) |

Table 8 below provides examples of other applications for N5′ person-worn detector for aerosolized chemical threats.

| Detector | | N5's ChemBadgER |
|---|---|---|
| Applications | Use Case Examples | Solution |
| Environmental | Safe cities; real-time | Widely distributed |
| Air Quality | detection of toxic chemicals | fixed-site monitor |
| | and aerosol burden; acid rain | |
| Health care | Nursing homes; clinics | Low-cost, person- |
| | | worn or fixed site |
| Industrial | High pressure, liquid leak | Low-cost, fixed site |

Embodiments herein include a device which detects aerosols only (e.g., NTAs and PBAs), as well as a device which detects both aerosols and vapors (e.g., CWAs and TICs).

The aerosols can be in liquid or solid form, which are collected, converted to a gas phase, and which are then detected by the sensors. In contrast to current technologies, current aerosol detectors are a massive size and cannot be worn or handheld. In embodiments herein, for example, the microheater captures small particles (i.e., minute amount) on the microheater's capture area. A rapid temperature then vaporizes the sample collected in the capture area, so that the sample then becomes a gas, for analysis by the SPH sensor array. The SPH sensor array can use six die sensors, for example, instead of one. Each of the six die sensors comprise two to four unique elements, which generates a heat map. The system will analyze the substance and let the user know the chemical makeup of the aerosol, such as a chemical warfare agent, fentanyl, etc. For example, the data can then be analyzed and displayed to a user wearing the device. If multiple users are each wearing a sensing device in the same area, for example, the information can be relayed from one of those users to the other users in the area to notify them of any detected aerosols. This could prevent multiple users from being exposed to high levels of contaminants (e.g., fentanyl). Alternatively, this information can be relayed to a computing device (e.g., an edge cloud) where AI/machine learning is used to identify the aerosol, for data gathering, and for future analysis. AI/ML is used for the identification of the aerosol chemical makeup using pattern recognition from the large array of chemical sensor dies used in these systems. The information can further be relayed from the sensing devices to a computing device, such as at an agency or with a team leader to notify them of sensed contaminants. Although embodiments herein discuss CWAs, TICs, NTAs, and PBAs, the sensing device can be modified and used to detect any type of respiratory threat (e.g., biological, nuclear, radiological, and explosives).

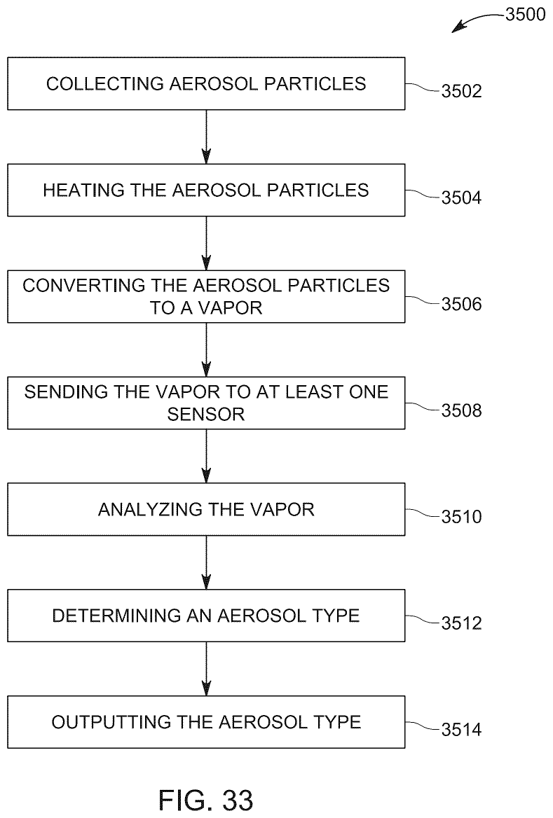

FIG. 33 illustrates a method 3500 for detecting an aerosol, wherein the method comprises: collecting aerosol particles 3502; heating the aerosol particles 3504; converting the aerosol particles to a vapor 3506; sending the vapor to at least one sensor 3508; analyzing the vapor 3510; and determining an aerosol type 3512. The method can also comprise outputting 3514, via a display for example, the aerosol type to a user. The display could also provide the amount of the substance detected, as well as an emergency warning.

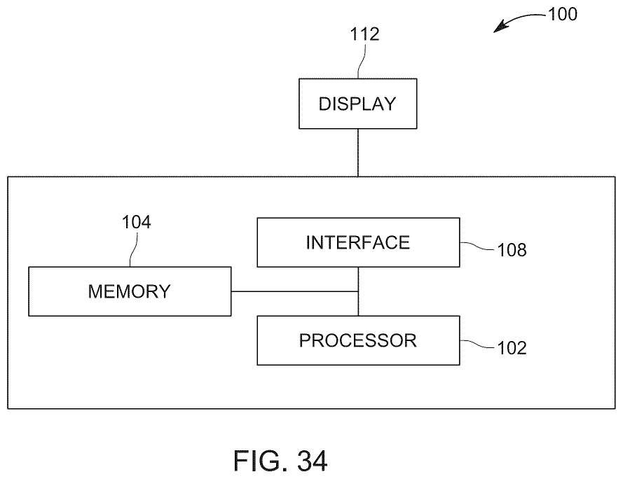

FIG. 34 shows a device 100 which can be used to execute instructions of methods disclosed herein, for example, collecting, analyzing, and processing information associated with detecting aerosol particles. The device 100 can include, for example, a processor 102, a memory 104, an interface 108, and a display 112. The processor 102 can be an ASIC such as discussed above. The interface 108 may be used for communicating with other portions of a computer, other components of an electronic device or external devices, including sending and receiving data. For example, the interface 108 can communicate with an aerosol collector 103. The processor 102 may be a combination of one or more of a microprocessor, controller, microcontroller, central processing unit, digital signal processor, application specific integrated circuit, field programmable gate array, or any other suitable computing device, resource, or combination of hardware, software and/or encoded logic operable to provide, either alone or in conjunction with other components, such as the primary and secondary memory. For example, the processor 102 may execute instructions stored in the memory 104. The display 112 can, for example, show an output associated with the detected aerosol particles.

The memory 104 may include any form of volatile or non-volatile computer readable memory including, without limitation, persistent storage, solid state memory, remotely mounted memory, magnetic media, optical media, RAM, read-only memory (ROM), removable media, or any other suitable local or remote memory component. The memory 104 may store any suitable instructions, data or information, including software and encoded logic, utilized by a computer and/or other electronic device. The memory 104 may be used to store any calculations made by processor 102 and/or any data received via interface 108.

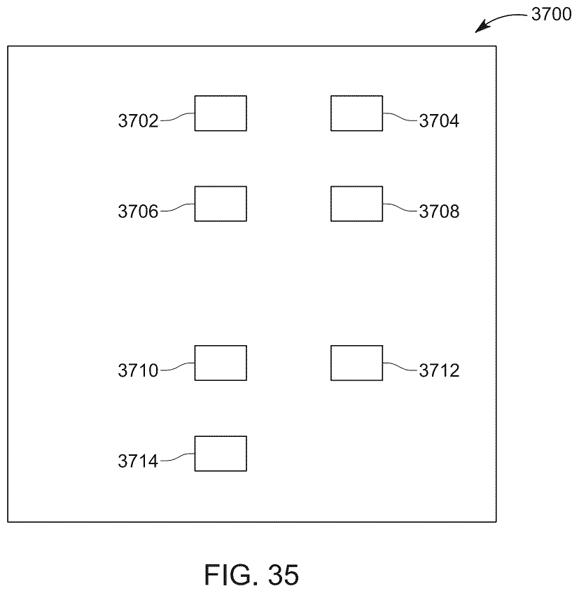

FIG. 35 illustrates an embodiment of a device 3700 for detecting aerosols. The device 3700, e.g., a sensing device, can be worn by a user, held in a user's hand, or mounted to another apparatus, such as a pole or a drone. The device 3700 includes at least an aerosol collector 3702 for collecting the aerosol particles, a display 3705, a microheater 3704, a Semi-conductor Photocatalytic Hybrid (SPH) array die 3706 containing at least one sensor, and a UV LED die 3707. In addition, the device can comprise an ASIC 3712, microprocessor, or microcontroller for controlling the device, including power, sending and receiving data, as well as analyzing the data. The sensing device can communicate with other sensing devices or a cloud computing device to relay information about the detected aerosol, via an interface 3714.

The disclosed embodiments provide methods, systems, and devices for a detecting aerosol particles. It should be understood that this description is not intended to limit the invention. On the contrary, the embodiments are intended to cover alternatives, modifications and equivalents, which are included in the spirit and scope of the invention. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein. The methods or flowcharts provided in the present application may be implemented in a computer program, software or firmware tangibly embodied in a computer-readable storage medium for execution by a specifically programmed computer or processor.

What is claimed is:

1. A method for detecting an aerosol, wherein the method comprises:
collecting aerosol particles;
heating the aerosol particles;
converting the aerosol particles to a vapor;
sending the vapor to at least one sensor;
analyzing the vapor; and
determining an aerosol type.

2. The method according to claim 1, wherein the aerosol is a suspension of fine solid particles or liquid droplets in air or in another gas and the vapor is a gas.

3. The method according to claim 1, wherein the method further comprises:
displaying the aerosol type to a user.

4. The method according to claim 1, wherein the aerosol type is a non-traditional agent (NTA) or a pharmaceutical-based agent (PBA).

5. The method according to claim 1, wherein the aerosol particles are collected by a microchannel collector.

6. The method according to claim 1, wherein the aerosol particles are collected by a microwell collector.

7. The method according to claim 1, wherein the aerosol particles are heated and desorbed by a microheater.

8. The method according to claim 1, wherein the vapor is analyzed using machine learning.

9. The method according to claim 1, wherein the aerosol particles are a liquid or a solid.

10. A device for detecting an aerosol, wherein the device comprises:
an aerosol collector to collect aerosol particles;
a heater to heat the aerosol particles;
and a computer processor configured to:
convert the aerosol particles to a vapor;
send the vapor to at least one sensor;
analyze the vapor; and
determine an aerosol type.

11. The device according to claim 10, wherein the aerosol is a suspension of fine solid particles or liquid droplets in air or in another gas and the vapor is a gas.

12. The device according to claim 10, wherein the device further comprises:
a display for displaying the aerosol type to a user.

13. The device according to claim 10, wherein the aerosol type is a non-traditional agent (NTA) or a pharmaceutical-based agent (PBA).

14. The device according to claim 10, wherein the aerosol collector is a microchannel collector.

15. The device according to claim 10, wherein the aerosol collector is a microwell collector.

16. The device according to claim 10, wherein the aerosol particles are heated and desorbed by a microheater.

17. The device according to claim 10, wherein the vapor is analyzed using machine learning.

18. The device according to claim 10, wherein the aerosol particles are a liquid or a solid.

19. The device according to claim 10, wherein the device is wearable by a user and is no larger than 8 cm×5 cm×1 cm, with a weight of less than 100 grams.

20. The device according to claim 10, wherein the device is mounted to an apparatus.
